SEQUENCE LISTING (23 sequences)

SEQ ID NO: 1 - DNA, 8465 bp, Artificial Sequence
  Left TALEN
  CDS (5131)..(8433)
agccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtttccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaagcttcttgttctttttgcagaagctcagaataaacgctcaactttggcctcgaggccaccatggcttcctcccctccaaagaaaaagagaaaggttgcggccgctgactacaaggatgacgacgataaaagttggaaggacgcaagtggttggtctagaatgcatgcggccccgcgacggcgtgctgcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcgctcacgggagcacccctcaacctgaccccagaccaggttgtggccatcgccagcaacataggtggcaagcaggccctcgaaaccgtccagagactgttaccggttctctgccaggaccacggcctgaccccggaacaggtggttgcaatcgcgtcacacgatgggggaaagcaggccctagaaaccgttcagcgactcctgcccgtcctgtgccaggcccacggcctgacccccgaccaggttgtcgctattgctagtaacggcggaggcaaacaggcgctggaaacagttcagcgcctcttgccggtcttgtgtcaggcccacggcctgacccccgcccaggttgtcgctattgctagtaacggcggaggcaaacaggcgctggaaacagttcagcgcctcttgccggtcttgtgtcaggaccacggcctgaccccggaccaggtggttgcaatcgcgtcacacgatgggggaaagcaggccctagaaaccgttcagcgactcctgcccgtcctgtgccaggaccacggcctgacccccgaacaggttgtcgctattgctagtaacggcggaggcaaacaggcgctggaaacagttcagcgcctcttgccggtcttgtgtcaggcccacggcctgacccccgaccaggttgtcgctattgctagtaacggcggaggcaaacaggcgctggaaacagttcagcgcctcttgccggtcttgtgtcaggcccacggcctgaccccagcccaagttgtcgcgattgcaagcaacaacggaggcaaacaagccttagaaacagtccagagattgttgccggtgctgtgccaagaccacggcctgaccccggaccaggtggttgcaatcgcgtcacacgatgggggaaagcaggccctagaaaccgttcagcgactcctgcccgtcctgtgccaggaccacggcctgacccccgaacaggttgtcgctattgctagtaacggcggaggcaaacaggcgctggaaacagttcagcgcctcttgccggtcttgtgtcaggcccacggcctgaccccagaccaagttgtcgcgattgcaagcaacaacggaggcaaacaagccttagaaacagtccagagattgttgcctgtgctgtgccaagcccacggcctgaccccggcccaggtggttgcaatcgcgtcacacgatgggggaaagcaggccctagaaaccgttcagcgactcctgcccgtcctgtgccaggaccacggcctgaccccagaccaggttgtggccatcgccagcaacataggtggcaagcaggccctcgaaaccgtccagagactgttaccggttctctgccaggaccacggcctgaccccggaacaggtggttgcaatcgcgtcacacgatgggggaaagcaggccctagaaaccgttcagcgactcctgcccgtcctgtgccaggcccacggcctgacccccgaccaggttgtcgctattgctagtaacggcggaggcaaacaggcgctggaaacagttcagcgcctcttgccggtcttgtgtcaggcccacggcctgaccccagcccaagttgtcgcgattgcaagcaacaacggaggcaaacaagccttagaaacagtccagagattgttgccggtgctgtgccaagaccacggcctgaccccagaccaagttgtcgcgattgcaagcaacaacggaggcaaacaagccttagaaacagtccagagattgttgccggtgctgtgccaagaccacggcctgaccccagaacaagttgtcgcgattgcaagcaacaacggaggcaaacaagccttagaaacagtccagagattgttgccggtgctgtgccaagcccacggcctgaccccagaccaggttgtggccatcgccagcaacataggtggcaagcaggccctcgaaaccgtccagagactgttaccggttctctgccaggcccacggcctgacgcctgagcaggtagtggctattgcatccaacatagggggcagacccgcactggagtcaatcgtggcccagctttcgaggccggaccccgcgctggccgcactcactaatgatcatcttgtagcgctggcctgcctcggcggacgtcctgccatggatgcagtgaaaaagggattgccgcacgcgccggaattgatcagatcccagctagtgaaatctgaattggaagagaagaaatctgaacttagacataaattgaaatatgtgccacatgaatatattgaattgattgaaatcgcaagaaattcaactcaggatagaatccttgaaatgaaggtgatggagttctttatgaaggtttatggttatcgtggtaaacatttgggtggatcaaggaaaccagacggagcaatttatactgtcggatctcctattgattacggtgtgatcgttgatactaaggcatattcaggaggttataatcttccaattggtcaagcagatgaaatgcaaagatatgtcgaagagaatcaaacaagaaacaagcatatcaaccctaatgaatggtggaaagtctatccatcttcagtaacagaatttaagttcttgtttgtgagtggtcatttcaaaggaaactacaaagctcagcttacaagattgaatcatatcactaattgtaatggagctgttcttagtgtagaagagcttttgattggtggagaaatgattaaagctggtacattgacacttgaggaagtgagaaggaaatttaataacggtgagataaacttttaaaaaatcagcctcgactgtgccttctagttgcc

SEQ ID NO: 2 - protein, 1100 aa, Artificial Sequence
  Synthetic Construct
MASSPPKKKRKVAAADYKDDDDKSWKDASGWSRMHAAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNNGGKQALETVQRLLPVLCQDHGLTPDQVVAIASNNGGKQALETVQRLLPVLCQDHGLTPEQVVAIASNNGGKQALETVQRLLPVLCQAHGLTPDQVVAIASNIGGKQALETVQRLLPVLCQAHGLTPEQVVAIASNIGGRPALESIVAQLSRPDPALAALTNDHLVALACLGGRPAMDAVKKGLPHAPELIRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 3 - DNA, 8159 bp, Artificial Sequence
  Right TALEN
  CDS (5131)..(8127)
agccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtttccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaagcttcttgttctttttgcagaagctcagaataaacgctcaactttggcctcgaggccaccatggcttcctcccctccaaagaaaaagagaaaggttgcggccgctgactacaaggatgacgacgataaaagttggaaggacgcaagtggttggtctagaatgcatgcggccccgcgacggcgtgctgcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcgctcacgggagcacccctcaacctgaccccggaccaggtggttgcaatcgcgtcacacgatgggggaaagcaggccctagaaaccgttcagcgactcctgcccgtcctgtgccaggaccacggcctgacccccgaacaggttgtcgctattgctagtaacggcggaggcaaacaggcgctggaaacagttcagcgcctcttgccggtcttgtgtcaggcccacggcctgaccccggaccaggtggttgcaatcgcgtcacacgatgggggaaagcaggccctagaaaccgttcagcgactcctgcccgtcctgtgccaggcccacggcctgaccccagcccaggttgtggccatcgccagcaacataggtggcaagcaggccctcgaaaccgtccagagactgttaccggttctctgccaggaccacggcctgacccccgaccaggttgtcgctattgctagtaacggcggaggcaaacaggcgctggaaacagttcagcgcctcttgccggtcttgtgtcaggaccacggcctgaccccggaacaggtggttgcaatcgcgtcacacgatgggggaaagcaggccctagaaaccgttcagcgactcctgcccgtcctgtgccaggcccacggcctgacccccgaccaggttgtcgctattgctagtaacggcggaggcaaacaggcgctggaaacagttcagcgcctcttgccggtcttgtgtcaggcccacggcctgaccccggcccaggtggttgcaatcgcgtcacacgatgggggaaagcaggccctagaaaccgttcagcgactcctgcccgtcctgtgccaggaccacggcctgaccccggaccaggtggttgcaatcgcgtcacacgatgggggaaagcaggccctagaaaccgttcagcgactcctgcccgtcctgtgccaggaccacggcctgaccccggaacaggtggttgcaatcgcgtcacacgatgggggaaagcaggccctagaaaccgttcagcgactcctgcccgtcctgtgccaggcccacggcctgaccccagaccaagttgtcgcgattgcaagcaacaacggaggcaaacaagccttagaaacagtccagagattgttgcctgtgctgtgccaagcccacggcctgacccccgcccaggttgtcgctattgctagtaacggcggaggcaaacaggcgctggaaacagttcagcgcctcttgccggtcttgtgtcaggaccacggcctgaccccagaccaagttgtcgcgattgcaagcaacaacggaggcaaacaagccttagaaacagtccagagattgttgccggtgctgtgccaagaccacggcctgaccccagaacaggttgtggccatcgccagcaacataggtggcaagcaggccctcgaaaccgtccagagactgttaccggttctctgccaggcccacggcctgaccccagaccaagttgtcgcgattgcaagcaacaacggaggcaaacaagccttagaaacagtccagagattgttgcctgtgctgtgccaagcccacggcctgaccccggcccaggtggttgcaatcgcgtcacacgatgggggaaagcaggccctagaaaccgttcagcgactcctgcccgtcctgtgccaggaccacggcctgacgcctgagcaggtagtggctattgcatccaacggagggggcagacccgcactggagtcaatcgtggcccagctttcgaggccggaccccgcgctggccgcactcactaatgatcatcttgtagcgctggcctgcctcggcggacgtcctgccatggatgcagtgaaaaagggattgccgcacgcgccggaattgatcagatcccagctagtgaaatctgaattggaagagaagaaatctgaacttagacataaattgaaatatgtgccacatgaatatattgaattgattgaaatcgcaagaaattcaactcaggatagaatccttgaaatgaaggtgatggagttctttatgaaggtttatggttatcgtggtaaacatttgggtggatcaaggaaaccagacggagcaatttatactgtcggatctcctattgattacggtgtgatcgttgatactaaggcatattcaggaggttataatcttccaattggtcaagcagatgaaatgcaaagatatgtcgaagagaatcaaacaagaaacaagcatatcaaccctaatgaatggtggaaagtctatccatcttcagtaacagaatttaagttcttgtttgtgagtggtcatttcaaaggaaactacaaagctcagcttacaagattgaatcatatcactaattgtaatggagctgttcttagtgtagaagagcttttgattggtggagaaatgattaaagctggtacattgacacttgaggaagtgagaaggaaatttaataacggtgagataaacttttaaaaaatcagcctcgactgtgccttctagttgcc

SEQ ID NO: 4 - protein, 998 aa, Artificial Sequence
  Synthetic Construct
MASSPPKKKRKVAAADYKDDDDKSWKDASGWSRMHAAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNNGGKQALETVQRLLPVLCQAHGLTPAQVVAIASNGGGKQALETVQRLLPVLCQDHGLTPDQVVAIASNNGGKQALETVQRLLPVLCQDHGLTPEQVVAIASNIGGKQALETVQRLLPVLCQAHGLTPDQVVAIASNNGGKQALETVQRLLPVLCQAHGLTPAQVVAIASHDGGKQALETVQRLLPVLCQDHGLTPEQVVAIASNGGGRPALESIVAQLSRPDPALAALTNDHLVALACLGGRPAMDAVKKGLPHAPELIRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 5 - DNA, 4860 bp, Artificial Sequence
  donor vector
  CDS (98)..(817)
cgccattctgcctggggacgtcggagcaagcttgatttaggtgacactatagaatacaagctacttgttctttttgcaggatcccatcgatgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggcgcgccgcggcgccaatgcattgggcccggtacccagcttttgttccctttagtgagggttaatacttcttgctgcactgggaattcagaaaacatgagagctcacgggagatgagtgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgatcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggcaagtacgccaagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaac

SEQ ID NO: 6 - protein, 239 aa, Artificial Sequence
  Synthetic Construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 7 - DNA, 45 bp, Artificial Sequence
  Xltyr-CMVEGFP-F
aacatgagagctcacgggagatgagtgcgcgcttggcgtaatcat

SEQ ID NO: 8 - DNA, 47 bp, Artificial Sequence
  Xltyr-CMVEGFP-R
ttctgaattcccagtgcagcaagaagtattaaccctcactaaaggga

SEQ ID NO: 9 - DNA, 25 bp, Artificial Sequence
  tyr-genomic F
ggagaggatggcctctggagagata

SEQ ID NO: 10 - DNA, 24 bp, Artificial Sequence
  tyr-genomic R
ggtgggatggattcctcccagaag

SEQ ID NO: 11 - DNA, 25 bp, Artificial Sequence
  pCS2-F
ataagatacattgatgagtttggac

SEQ ID NO: 12 - DNA, 25 bp, Artificial Sequence
  pCS2-R
atgcagctggcacgacaggtttccc

SEQ ID NO: 13 - DNA, 25 bp, Artificial Sequence
  oligonucleotide 13
caccgctctcacaggccacccccca

SEQ ID NO: 14 - DNA, 25 bp, Artificial Sequence
  oligonucleotide 14
aaactggggggtggcctgtgagagc

SEQ ID NO: 15 - DNA, 25 bp, Artificial Sequence
  oligonucleotide 15
caccgtggatccgtggggtggcccc

SEQ ID NO: 16 - DNA, 25 bp, Artificial Sequence
  oligonucleotide 16
aaacggggccaccccacggatccac

SEQ ID NO: 17 - DNA, 24 bp, Artificial Sequence
  oligonucleotide 17
caccggtgcctgaccaaggtgccc

SEQ ID NO: 18 - DNA, 24 bp, Artificial Sequence
  oligonucleotide 18
aaacgggcaccttggtcaggcacc

SEQ ID NO: 19 - DNA, 28 bp, Artificial Sequence
  primer 19
acaccaagacagacatctctgtcccttg

SEQ ID NO: 20 - DNA, 22 bp, Artificial Sequence
  primer 20
atccgtatccaatgtggggaac

SEQ ID NO: 21 - DNA, 22 bp, Artificial Sequence
  primer 21
ccgcaacctccccttctacgag

SEQ ID NO: 22 - DNA, 23 bp, Artificial Sequence
  primer 22
tcagcaggtcaaggggaggaatg

SEQ ID NO: 23 - DNA, 5966 bp, Artificial Sequence
  donor vector
ctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagttattaatagtaatcaattacggggtcattagttcatagcccatatatggagttccgcgttacatacccggggccaccccacggatccatggtgagtaagggagaggaagataatatggcctcccttcccgctacgcacgaactccacatcttcgggtcaatcaacggtgttgacttcgacatggtgggccagggcaccggcaatcccaatgacggatacgaagaactcaatttgaagagtacaaagggcgatctccaattctcaccttggattctggttccccacattggatacggatttcatcagtacctgccgtaccccgatgggatgagcccatttcaggctgcaatggtagatggtagcggttaccaagtacaccgaactatgcaatttgaggacggtgcctcactgacagtgaactatcggtatacttacgaaggaagccacatcaagggagaggcacaggtcaaaggaaccggatttccagccgacgggccagtcatgacaaactccctgaccgccgcagattggtgccgcagcaaaaagacctatccaaatgacaagaccattatctcgacattcaaatggagctacaccaccggaaacggcaaacgctatcggtctaccgccaggacaacctacacatttgcaaaacctatggccgcaaactatctgaaaaaccagccgatgtatgtgttccgaaagacggaattaaaacactcgaaaacagaactaaactttaaagagtggcagaaagcctttaccgacgtaatgggcatggacgagctgtataagggaagcggagagggcagaggaagtctgctaacatgcggtgacgtcgaggagaatcctggacctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaccaaggtgcccgggtctagaatgctgatgggctagcaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtttccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttg

(19) United States
(12) Patent Application Publication
(10) Pub. No.: US 2019/0177745 A1
(43) Pub. Date: Jun. 13, 2019

(54) Vector for Nucleic Acid Insertion

(71) Applicant: HIROSHIMA UNIVERSITY, Hiroshima (JP)

(72) Inventors: Takashi Yamamoto, Hiroshima (JP); Kenichi Suzuki, Hiroshima (JP); Tetsushi Sakuma, Hiroshima (JP); Yuto Sakane, Hiroshima (JP)

(73) Assignee: HIROSHIMA UNIVERSITY, Hiroshima (JP)

(21) Appl. No.: 16/283,033

(22) Filed: Feb. 22, 2019

Related U.S. Application Data
(62) Division of application No. 15/032,544, filed on Apr. 27, 2016.

(30) Foreign Application Priority Data
Nov. 6, 2013 (JP) 2013-230349

(51) Int. Cl. C12N 15/85; C12N 15/90; C12N 15/10

(52) U.S. Cl. CPC C12N 15/8509; C12N 15/90; A01K 2227/50; C12N 2510/00; A01K 2217/00; C12N 15/102

(57) ABSTRACT

The present invention provides the following: a vector for inserting a desired nucleic acid into a predetermined site of a nucleic acid comprising a region formed of a first nucleotide sequence, the predetermined site, and a region composed of a second nucleotide sequence, in the stated order in the 5′-to-3′ direction, wherein the vector comprises a region formed of the first nucleotide sequence, the desired nucleic acid, and the second nucleotide sequence in the stated order in the 5′-to-3′ direction; a kit that includes this vector; a method of inserting a nucleic acid comprising a step for introducing this vector into a cell; a cell acquired by this method; and an organism comprising this cell.

### SEQUENCE LISTING SUBMISSION VIA EFS-WEB

A computer readable text file, entitled “SequenceListing.txt,” created on or about Apr. 26, 2016 with a file size of about 82 kb contains the sequence listing for this application and is hereby incorporated by reference in its entirety.

### TECHNICAL FIELD

The present invention relates to a method for inserting a desired nucleic acid into a predetermined site in a nucleic acid contained in a cell, a vector for the method, a kit for the method, and a cell obtained by the method. Further, the present invention relates to an organism comprising a cell containing a desired nucleic acid and a method for producing the organism.

### BACKGROUND ART

TALENs (TALE Nucleases), ZFNs (Zinc Finger Nucleases), and the like are known as polypeptides including a plurality of nuclease subunits formed of DNA binding domains and DNA cleavage domains (Patent Literatures 1 to 4 and Non-Patent Literature 1). As for these artificial nucleases, a plurality of adjacent DNA cleavage domains form multimers at each binding site of the DNA binding domains, and thereby catalyze double strand break of DNAs. Each of the DNA binding domains contains repeats of a plurality of DNA binding modules. Each of the DNA binding modules recognizes a specific base pair in the DNA strand. Accordingly, a specific nucleotide sequence can be specifically cleaved by appropriately designing a DNA binding module. Other known nucleases which specifically cleave the specific nucleotide sequence are an RNA-guided nuclease such as a CRISPR/Cas system (Non-Patent Literature 2) and an RNA-guided FokI nuclease with a FokI nuclease fused to the CRISPR/Cas system (FokI-dCas9) (Non-Patent Literature 3). Various genetic modifications such as gene deletion and insertion on a genomic DNA and mutation introduction are performed using errors and recombination during repair of breaks by these nucleases (refer to Patent Literatures 5 to 6 and Non-Patent Literature 4).

As methods for inserting a desired nucleic acid into a cell using an artificial nuclease, the methods described in Non-Patent Literatures 5 to 8 are known. Non-Patent Literature 5 describes a method for inserting a foreign DNA by homologous recombination using TALENs. Non-Patent Literature 6 describes a method for inserting a foreign DNA by homologous recombination using ZFNs. However, the vector used for homologous recombination is long-stranded and cannot be easily produced. Depending on the cells and organisms, the homologous recombination efficiency is sometimes low. Therefore, these methods can be used only for limited cells and organisms. In order to obtain a modified organism that stably has a cell with a desired nucleic acid inserted therein, it is effective to obtain an adult organism by introducing a target nucleic acid into an animal embryo and differentiating the embryo. However, the homologous recombination efficiency is low in the animal embryo, and thus these methods are inefficient. A known technique for introducing a foreign DNA into animal embryos is ssODN-mediated gene modification. In this technique, it is only possible to introduce a short DNA with about several 10 bp.

The method described in Non-Patent Literature 7 or 8 is a method for inserting a nucleic acid into a cell by using an artificial nuclease without using homologous recombination. Non-Patent Literature 7 discloses a method for inserting a foreign DNA by cleaving a nucleic acid in a cell and a foreign DNA to be inserted using the ZFNs and TALENs, and joining the cleaved sites of the nucleic acid and the foreign DNA by the action of non-homologous end joining (NHEJ). However, the method described in Non-Patent Literature 7 does not control the direction of the nucleic acid to be inserted, and the junction of the nucleic acid to be inserted is not accurate. In the method described in Non-Patent Literature 8, a single-stranded end formed from the nucleic acid in the cell by nuclease cleavage is joined to a single-stranded end formed from the foreign DNA by annealing them, in order to achieve the control of direction and accurate joining. However, the method described in Non-Patent Literature 8 requires use of heterodimeric ZFNs and heterodimeric TALENs in order to prevent a DNA after insertion from being cleaved again, and a highly-active homodimeric artificial nuclease cannot be used in this method. The method described in Non-Patent Literature 8 is not used to insert the desired nucleic acid into animal embryos. Further, in the method described in Non-Patent Literature 8, the single-stranded end is frequently annealed to a wrong site, and a cell in which a nucleic acid is accurately inserted is not frequently obtained. In this regard, Non-Patent Literatures 5 to 8 do not describe a method of using an RNA-guided nuclease such as a CRISPR/Cas system or an RNA-guided FokI nuclease such as FokI-dCas9.

### CITATION LIST

### Patent Literatures

Patent Literature 1: PCT International Publication No. WO 2011-072246
Patent Literature 2: PCT International Publication No. WO 2011-154393
Patent Literature 3: PCT International Publication No. WO 2011-159369
Patent Literature 4: PCT International Publication No. WO 2012-093833
Patent Literature 5: Japanese Patent Application National Publication (Laid-Open) No. 2013-513389
Patent Literature 6: Japanese Patent Application National Publication (Laid-Open) No. 2013-529083 Non-Patent Literatures
Non-Patent Literature 1: Nat Rev Genet. 2010 September; 11 (9): 636-46.
Non-Patent Literature 2: Nat Protoc. 2013 November; 8 (11): 2281-308.
Non-Patent Literature 3: Nat Biotechnol. 2014 June; 32 (6): 569-76.
Non-Patent Literature 4: Cell. 2011 Jul. 22; 146 (2): 318-31.
Non-Patent Literature 5: Nat Biotechnol. 2011 Jul. 7; 29 (8): 731-4.
Non-Patent Literature 6: Nat Biotechnol. 2009 September; 27 (9): 851-7.
Non-Patent Literature 7: Biotechnol Bioeng. 2013 March; 110 (3): 871-80.
Non-Patent Literature 8: Genome Res. 2013 March; 23 (3): 539-46.

### SUMMARY OF INVENTION

### Problems to be Solved by the Invention

Therefore, an object of the present invention includes to provide a method for inserting a desired nucleic acid into a predetermined site of a nucleic acid in each cell of various organisms accurately and easily without requiring any complicated step such as production of a long-stranded vector, the method also enables insertion of a relatively long-stranded nucleic acid and can be used in combination with the homodimeric nuclease including a DNA cleavage domain, the RNA-guided nuclease or the RNA-guided FokI nuclease.

### Means for Solving the Problems

The present inventors focused on a region formed of a first nucleotide sequence and a region formed of a second nucleotide sequence which sandwich a predetermined site in which a nucleic acid is to be inserted, and designed a nuclease that specifically cleaves a moiety including these regions included in a nucleic acid in a cell. Further, the present inventors designed a vector including a region formed of a first nucleotide sequence, a desired nucleic acid to be inserted and a region formed of a second nucleotide sequence in the stated order in the 5′-end to 3′-end direction. Then, the present inventors introduced the designed vector into the cell, allowed the nuclease to act on the cell, and thereby effected cleavage of the predetermined site in the nucleic acid in the cell. Further, they allowed the nuclease to act on the vector, resulting in production of a nucleic acid fragment including the region formed of the first nucleotide sequence, the desired nucleic acid and the region formed of the second nucleotide sequence in the stated order in the 5′-end to 3′-end direction. As a result, in the cell, the first nucleotide sequence in the nucleic acid in the cell and the first nucleotide sequence in the vector were joined by microhomology-mediated end joining (MMEJ), and the second nucleotide sequence in the nucleic acid in the cell and the second nucleotide sequence in the vector were joined by MMEJ. Accordingly, the desired nucleic acid was accurately inserted into the predetermined site of the nucleic acid of the cell. It was possible to perform the insertion step on relatively long-stranded nucleic acids of several kb or more. The used nuclease specifically cleaves the moiety including the region formed of the first nucleotide sequence and the region formed of the second nucleotide sequence in the nucleic acid in the cell before insertion. However, the linked nucleic acid does not include a part of the moiety because of insertion of the desired nucleic acid. Thus, the nucleic acid was not cleaved again by the nuclease present in the cell and was stably maintained, and insertion of the desired nucleic acid occurred at high frequency.

According to the method, the sequences are joined by microhomology-mediated end joining which functions in many cells. Consequently, a desired nucleic acid can be accurately inserted at high frequency into cells at the developmental stage or the like with low homologous recombination efficiency. The method can be applied to a wide range of organisms and cells. Further, according to the method, a vector for introducing a nuclease and a vector for inserting a nucleic acid can be simultaneously inserted into a cell and thus the operation is simple. Furthermore, according to the method, changes in the nucleic acid moiety in the cell due to microhomology-mediated end joining prevent the inserted nucleic acid from being cleaved again. As the DNA cleavage domain included in the nuclease, a highly active homodimeric domain can also be used, and a wide range of experimental materials can be selected.

That is, according to a first aspect of the present invention, there is provided a vector for inserting a desired nucleic acid into a predetermined site in a nucleic acid contained in a cell by a nuclease,

wherein the nucleic acid contained in the cell includes a region formed of a first nucleotide sequence, the predetermined site, and a region formed of a second nucleotide sequence in the stated order in a 5′-end to 3′-end direction,

wherein the nuclease specifically cleaves a moiety including the region formed of the first nucleotide sequence and the region formed of the second nucleotide sequence included in the cell, and

wherein the vector includes a region formed of a first nucleotide sequence, the desired nucleic acid, and a region formed of a second nucleotide sequence in the stated order in a 5′-end to 3′-end direction.

That is, according to a second aspect of the present invention, there is provided a vector for inserting a desired nucleic acid into a predetermined site in a nucleic acid contained in a cell by a nuclease including a first DNA binding domain and a second DNA binding domain,

wherein the nucleic acid contained in the cell includes a region formed of a first nucleotide sequence, the predetermined site, and a region formed of a second nucleotide sequence in the stated order in a 5′-end to 3′-end direction,

wherein the region formed of the first nucleotide sequence, the predetermined site and the region formed of the second nucleotide sequence in the nucleic acid contained in the cell are each located between a region formed of a nucleotide sequence recognized by the first DNA binding domain and a region formed of a nucleotide sequence recognized by the second DNA binding domain,

wherein the vector includes a region formed of a first nucleotide sequence, the desired nucleic acid, and a region formed of a second nucleotide sequence in the stated order in a 5′-end to 3′-end direction,

wherein the region formed of the first nucleotide sequence and the region formed of the second nucleotide sequence in the vector are each located between a region formed of a nucleotide sequence recognized by the first DNA binding domain and a region formed of a nucleotide sequence recognized by the second DNA binding domain, and

wherein the vector produces a nucleic acid fragment including the region formed of the first nucleotide sequence, the desired nucleic acid, and the region formed of the second nucleotide sequence in the stated order in the 5′-end to 3′-end direction by the nuclease.

Further, according to a third aspect of the present invention, there is provided the vector according to the first or second aspect, wherein the first nucleotide sequence in the nucleic acid contained in the cell and the first nucleotide sequence in the vector are joined by microhomology-mediated end joining (MMEJ), and the second nucleotide sequence in the nucleic acid contained in the cell and the second nucleotide sequence in the vector are joined by MMEJ, whereby the desired nucleic acid is inserted.

Further, according to a fourth aspect of the present invention, there is provided the vector according to the second aspect, wherein the nuclease is a homodimeric nuclease and the vector is a circular vector.

Further, according to a fifth aspect of the present invention, there is provided the vector according to the first aspect, wherein the nuclease is a Cas9 nuclease.

Further, according to a sixth aspect of the present invention, there is provided the vector according to the second aspect, wherein the nuclease is a TALEN.

Further, according to a seventh aspect of the present invention, there is provided a kit for inserting a desired nucleic acid into a predetermined site in a nucleic acid contained in a cell, comprising the vector according to any one of the first to sixth aspects and a vector for expressing a nuclease.

Further, according to an eighth aspect of the present invention, there is provided a method for inserting a desired nucleic acid into a predetermined site in a nucleic acid contained in a cell, including a step of introducing the vector according to any one of the first to sixth aspects and a vector for expressing a nuclease into a cell.

Further, according to a ninth aspect of the present invention, there is provided a cell obtained by the method according to the eighth aspect.

Further, according to a tenth aspect of the present invention, there is provided an organism comprising the cell according to the ninth aspect.

Further, according to an eleventh aspect of the present invention, there is provided a method for producing an organism comprising a desired nucleic acid, comprising a step of differentiating a cell obtained by the method according to the eighth aspect.

Further, according to a twelfth aspect of the present invention, there is provided an organism produced by the method according to the eleventh aspect.

### Effects of the Invention

When the vector of the present invention is used, a desired nucleic acid can be accurately and easily inserted into a predetermined site of a nucleic acid in each cell of various organisms without requiring any complicated step such as production of a long-stranded vector, without depending on homologous recombination efficiency in cells or organisms, and without causing any frame shift. Relatively long-stranded nucleic acids of several kb or more can also be inserted. The method for inserting a nucleic acid using the vector of the present invention can be used in combination with a nuclease including a homodimeric DNA cleavage domain with high nuclease activity. Alternatively, the method for inserting a nucleic acid using the vector of the present invention can be used in combination with an RNA-guided nuclease such as a CRISPR/Cas system. Further, when the vector of the present invention is used, it is possible to accurately design a junction and to knock-in a functional domain with in-frame. Thus, when a nucleic acid containing a gene as a label is used, the organism subjected to target insertion can be easily identified by detecting expression of the gene. It is possible to easily obtain an organism with a desired nucleic acid inserted therein at high frequency. Further, the method for inserting a nucleic acid using the vector of the present invention can be used for undifferentiated cells such as animal embryos with low homologous recombination efficiency. Consequently, by inserting a desired nucleic acid into an undifferentiated cell using the vector of the present invention and differentiating the obtained undifferentiated cell, it is possible to easily obtain an adult organism that stably maintains the desired nucleic acid.

### BRIEF DESCRIPTION OF DRAWINGS

The patent or application file contains at least one drawing executed in color. Copies of this patent or patent application publication with color drawings will be provided by the Office upon request and payment of the necessary fee.

FIG. 1 is a schematic view illustrating target integration to a tyr locus in the case where the whole vector containing a desired nucleic acid is inserted using TALENs.

FIG. 2 is a schematic view illustrating the case where a part of the vector containing a desired nucleic acid is inserted using TALENs.

FIG. 3 is a schematic view of the design of the vector of the present invention using a CRISPR/Cas system.

FIG. 4a is a schematic view of a case where the whole vector containing a desired nucleic acid is inserted using a CRISPR/Cas system.

FIG. 4b is a schematic view of a case where the whole vector containing a desired nucleic acid is inserted using a CRISPR/Cas system.

FIG. 5 is a schematic view of a case where the whole vector containing a desired nucleic acid is inserted using a FokI-dCas9.

FIG. 6 illustrates phenotype of each embryonic into which TALENs and a vector for target integration (TAL-PITCh vector) have been microinjected. FIG. 6 illustrates bright field images (upper row) and GFP fluorescence images (lower row) of TALEN R+vector-injected embryos (negative control group; A) and TALEN mix+vector-injected embryos (experimental group; B).

FIG. 7 illustrates percentages of phenotypes in the negative control group and the experimental group. The phenotypes are classified into four groups (Full, Half, Mosaic and Non), except for abnormal embryo (gray, Abnormal). The number of individuals is shown at the top of each graph.

FIG. 8 illustrates detection of the introduction of the donor vector (TAL-PITCh vector) into a target gene locus. The lower views are photographs of electrophoresis of PCR products using primer sets at the upstream and downstream of a target sequence of tyrTALEN, and the sides of the vector. The upper view illustrates the positions of the primers. Each of the arrows in the lower views indicate a band that shows integration of each vector. The numeric characters correspond to individual numbers of FIG. 6.

FIGS. 9A and 9B illustrate sequence analysis of the junction between the insertion site and the donor vector (TAL-PITCh vector). The results of sequencing of PCR products (at the 5′-side and the 3′-side in FIG. 8) derived from Nos. 3 and 4 (FIG. 6) are shown. Sequences expected in MMEJ-dependent introduction are shown in the upper row. TALEN target sequences are underlined. Boxes near the center represent a spacer surrounding sequence shortened by MMEJ at the 5′-side and a spacer surrounding sequence shortened by MMEJ at the 3′-side, respectively. Each deletion is indicated by a dashed line (-), and each insertion is indicated by italics.

FIG. 10 is a schematic view of target integration to an FBL locus of a HEK293T cell using a CRISPR/Cas system.

FIGS. 11A and 11B illustrate the full length sequence of a donor vector (CRIS-PITCh vector). A mNeonGreen coding sequence is indicated in green, a 2A peptide coding sequence is indicated in purple and a puromycin resistance gene coding sequence is indicated in blue. A gRNA target sequence at the 5′-side and a gRNA target sequence at the 3′-side are underlined.

FIG. 11B is a continuation of FIG. 11A.

FIG. 12 is a mNeonGreen fluorescence image showing a phenotype of a HEK293T cell in which a vector expressing three types of gRNAs and Cas9 and a donor vector (CRIS-PITCh vector) have been co-introduced.

FIG. 13 illustrates sequence analysis of the junction between the insertion site and the donor vector (CRIS-PITCh vector). The sequences expected in MMEJ-dependent introduction are shown in the upper row. Each deletion is indicated by a dashed line (-), each insertion is indicated by a double underline, and each substitution is indicated by an underline.

### MODES FOR CARRYING OUT THE INVENTION

The vector provided by the first aspect of the present invention is a vector for inserting a desired nucleic acid into a predetermined site in a nucleic acid contained in a cell. Examples of the nucleic acid contained in the cell include genomic DNA in a cell. Examples of the cell origin include human; non-human mammals such as cow, miniature pig, pig, sheep, goat, rabbit, dog, cat, guinea pig, hamster, mouse, rat and monkey; birds; fish such as zebrafish; amphibia such as frog; reptiles; insects such as drosophila; and crustacea. Examples of the cell origin include plants such as Arabidopsis thaliana. The cell may be a cultured cell. The cell may be an immature cell, such as a pluripotent stem cell including an embryonic stem cell (ES cell) and an induced pluripotent stem cell (iPS cell), capable of differentiating into a more mature tissue cell. The embryonic stem cell and induced pluripotent stem cell can infinitely increase, and are useful as supply sources for a large amount of functional cells.

The cell into which the vector of the first aspect of the present invention is inserted includes a nucleic acid including a region formed of a first nucleotide sequence, a predetermined site in which a nucleic acid is to be inserted and a region formed of a second nucleotide sequence in the stated order in the 5′-end to 3′-end direction. The first nucleotide sequence and the second nucleotide sequence are expedient terms showing a relationship with the sequence included in the vector to be inserted. The first and second nucleotide sequences may be adjacent to the predetermined site directly or through a region consisting of a specific base sequence. When the first and second nucleotide sequences are adjacent to the predetermined site through the region consisting of a specific base sequence, the specific base sequence is preferably from 1 to 7 bases in length and more preferably from 1 to 3 bases in length. The first nucleotide sequence is preferably from 3 to 10 bases in length, more preferably from 4 to 8 bases in length, and even more preferably from 5 to 7 bases in length. The second nucleotide sequence is preferably from 3 to 10 bases in length, more preferably from 4 to 8 bases in length, and even more preferably from 5 to 7 bases in length.

The vector provided by the first aspect of the present invention is a vector for inserting a desired nucleic acid into a predetermined site in a nucleic acid contained in a cell using a nuclease. In the first aspect of the present invention, the nuclease specifically cleaves the moiety including the region formed of the first nucleotide sequence and the region formed of the second nucleotide sequence in the cell. Such a nuclease is, for example, a nuclease including a first DNA binding domain and a second DNA binding domain. This nuclease will be described in the section herein in which the vector provided by the second aspect of the present invention is described. Examples of another nuclease which performs the specific cleavage as described above include RNA-guided nucleases such as nucleases based on the CRISPR/Cas system. In the CRISPR/Cas system, a moiety called “PAM” is essential to cleave a double strand by the Cas9 nuclease. Examples of the Cas9 nuclease include SpCas9 derived from Streptococcus pyogenes and StCas9 derived from Streptococcus thermophilus. The PAM of SpCas9 is a “5′-NGG-3′” sequence (N represents any nucleotide) and a position where the double strand is cleaved is located at a position 3 bases upstream (at the 5′-end) of the PAM. A guide RNA (gRNA) in the CRISPR/Cas system recognizes a base sequence located at the 5′-side of the position where the double strand is cleaved. Then, the position where the double strand is cleaved in the CRISPR/Cas system corresponds to the predetermined site for inserting the desired nucleic acid in the nucleic acid contained in the cell. The region formed of the first nucleotide sequence and the region formed of the second nucleotide sequence are present at both ends of the predetermined site. Accordingly, the CRISPR/Cas system using the gRNA which recognizes the base sequence located at the 5′-end of the PAM contained in a nucleic acid in a cell can specifically cleave the moiety including the region formed of the first nucleotide sequence and the region formed of the second nucleotide sequence.

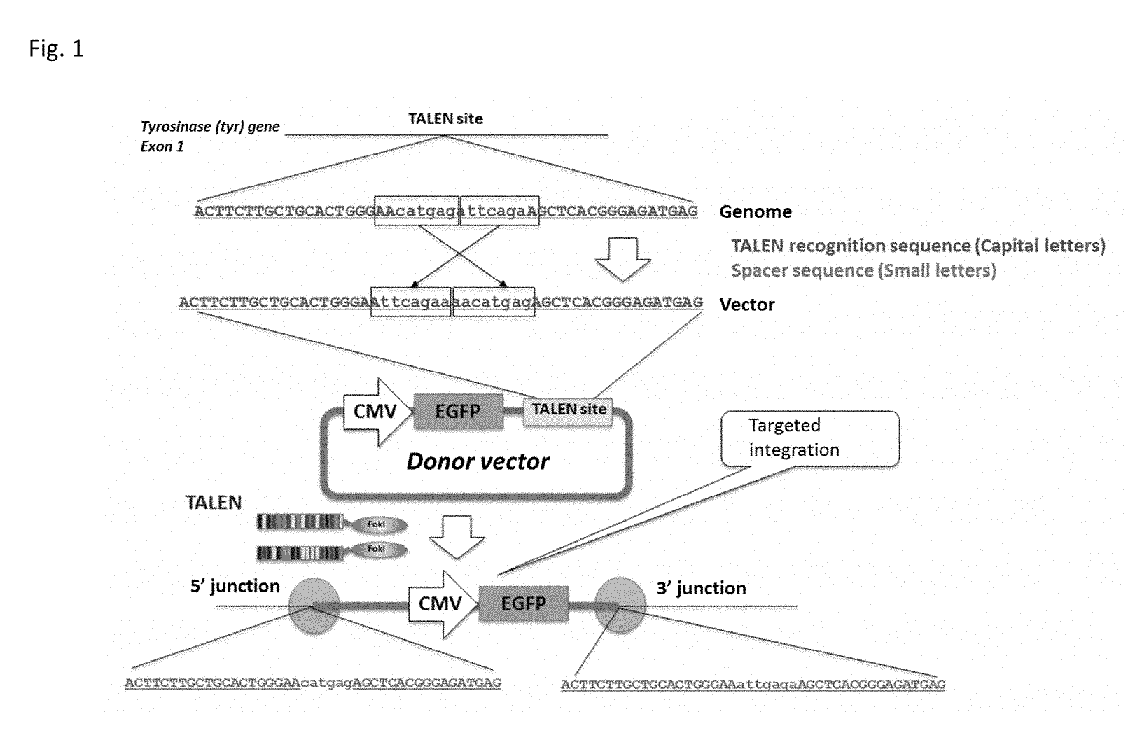

The vector provided by the first aspect of the present invention includes a region formed of a first nucleotide sequence, a desired nucleic acid to be inserted into a cell and a region formed of a second nucleotide sequence in the stated order in the 5′-end to 3′-end direction. The region formed of the first nucleotide sequence included in the vector is the same as the region formed of the first nucleotide sequence in the nucleic acid contained in the cell. The region formed of the second nucleotide sequence included in the vector is the same as the region formed of the second nucleotide sequence in the nucleic acid contained in the cell. A relationship between the first and second nucleotide sequences included in the vector and the first and second nucleotide sequences in the nucleic acid contained in the cell will be described using FIG. 1 as an example. “AAcatgag” contained in the TALEN site of FIG. 1 is a first nucleotide sequence. “AA” in the first nucleotide sequence is an overlap between the nucleotide sequence recognized by the first DNA binding domain and the first nucleotide sequence. “attcagaA” contained in the TALEN site of FIG. 1 is a second nucleotide sequence. The capital letter A of the second nucleotide sequence represents an overlap between the second nucleotide sequence and the nucleotide sequence recognized by the second DNA binding domain. On the other hand, “Attcagaa” contained in the donor vector of FIG. 1 is a second nucleotide sequence. The capital letter A included in the second nucleotide sequence represents an overlap between the nucleotide sequence recognized by the first DNA binding domain and the first nucleotide sequence. “aacatgag” contained in the donor vector of FIG. 1 is a first nucleotide sequence. A sequence encoding CMV and EGFP contained in the donor vector of FIG. 1 is a desired nucleic acid to be inserted into a cell. As illustrated in the schematic view of the donor vector of FIG. 1, the donor vector will be described by defining the region formed of the first nucleotide sequence (aacatgag) as the starting point. The donor vector of FIG. 1 includes a region formed of a first nucleotide sequence, a desired nucleic acid to be inserted into a cell and a region formed of the second nucleotide sequence in the stated order in the 5′-end to 3′-end direction. The donor vector of FIG. 1 will be described in comparison to the TALEN site of FIG. 1. In the TALEN site, the 3′-end of the first nucleotide sequence is adjacent to or in contact with the 5′-end of the second nucleotide sequence. On the other hand, in the donor vector, the 3′-end of the second nucleotide sequence is adjacent to or in contact with the 5′-end of the first nucleotide sequence. In this regard, in an example of FIG. 1, a positional relationship between the first nucleotide sequence and the second nucleotide sequence in the nucleic acid in the cell is reversed, compared to a positional relationship between the first nucleotide sequence and the second nucleotide sequence in the vector. Such a relationship results from the fact that the donor vector of FIG. 1 is a circular vector and the nuclease cleaves a moiety including the region formed of the first nucleotide sequence and the region formed of the second nucleotide sequence in the vector. Thus, the vector of the first aspect of the present invention is preferably a circular vector. In the case where the vector of the first aspect of the present invention is a circular vector, the 3′-end of the second nucleotide sequence and the 5′-end of the first nucleotide sequence which are contained in the vector of the first aspect of the present invention are preferably adjacent or directly linked to each other. In the case where the vector of the first aspect of the present invention is a circular vector and the second nucleotide sequence is adjacent to the first nucleotide sequence, the 3′-end of the second nucleotide sequence is separated from the 5′-end of the first nucleotide sequence preferably by a region of from 1 to 7 bases in length, more preferably from 1 to 5 bases in length, and even more preferably from 1 to 3 bases in length.

The vector provided by the second aspect of the present invention is a vector for inserting a desired nucleic acid using a nuclease including a first DNA binding domain and a second DNA binding domain.

Examples of the origin of a DNA binding domain include TALEs (transcription activator-like effectors) of plant pathogen Xanthomonas and Zinc fingers. Preferably, the DNA binding domain continuously includes one or more DNA binding modules that specifically recognize base pairs from the N-terminus. One DNA binding module specifically recognizes one base pair. Therefore, the first DNA binding domain and the second DNA binding domain each recognize a region formed of a specific nucleotide sequence. The nucleotide sequence recognized by the first DNA binding domain and the nucleotide sequence recognized by the second DNA binding domain may be the same as or different from each other. The number of DNA binding modules included in the DNA binding domain is preferably from 8 to 40, more preferably from 12 to 25, and even more preferably from 15 to 20, from the viewpoint of compatibility between the level of nuclease activity and the level of DNA sequence recognition specificity of the DNA cleavage domain. The DNA binding module is, for example, a TAL effector repeat. Examples of the length of a DNA binding module include a length of from 20 to 45, a length of from 30 to 38, a length of from 32 to 36 and a length of 34. All the DNA binding modules included in the DNA binding domain are preferably identical in length. The first DNA binding domain and the second DNA binding domain are preferably identical in origin and characteristics.

In the case where the RNA-guided FokI nuclease (FokI-dCas9) is used, the FokI-dCas9 forming a complex with a gRNA corresponds to the nuclease including the DNA binding domain in the second aspect. The dCas9 is a Cas9 whose catalytic activity is inactivated. The dCas9 is guided by a gRNA recognizing a base sequence located near the site in which a double strand is cleaved, and is linked to a nucleic acid. That is, the dCas9 forming a complex with a gRNA corresponds to the DNA binding domain in the second aspect.

The nuclease including the first DNA binding domain and the second DNA binding domain preferably includes a first nuclease subunit including a first DNA binding domain and a first DNA cleavage domain and a second nuclease subunit including a second DNA binding domain and a second DNA cleavage domain.

Preferably, the first DNA cleavage domain and the second DNA cleavage domain approach each other to form a multimer after each of the first DNA binding domain and the second DNA binding domain is linked to a DNA, and acquires an improved nuclease activity. The DNA cleavage domain is, for example, a DNA cleavage domain derived from a restriction enzyme FokI. The DNA cleavage domain may be a heterodimeric DNA cleavage domain or may be a homodimeric DNA cleavage domain. When the first DNA cleavage domain and the second DNA cleavage domain approach each other, a multimer is formed and an improved nuclease activity is obtained. However, In the case where neither the multimer is formed nor the improved nuclease activity is obtained even if the first DNA cleavage domain and the first DNA cleavage domain approach each other, and neither the multimer is formed nor the improved nuclease activity is obtained even if the second DNA cleavage domain and the second DNA cleavage domain approach each other, each of the first DNA cleavage domain and the second DNA cleavage domain is a heterodimeric DNA cleavage domain. In the case where a multimer is formed and the nuclease activity is improved when the first DNA cleavage domain and the first DNA cleavage domain approach each other, the first DNA cleavage domain is a homodimeric DNA cleavage domain. In the case of using the homodimeric DNA cleavage domain, a high nuclease activity is generally obtained. The first DNA cleavage domain and the second DNA cleavage domain are preferably identical in origin and characteristics.

In the case of using a TALEN, the first DNA binding domain and the first DNA cleavage domain in the first nuclease subunit are linked by a polypeptide consisting of from 20 to 70 amino acids, from 25 to 65 amino acids or from 30 to 60 amino acids, preferably from 35 to 55 amino acids, more preferably from 40 to 50 amino acids, even more preferably from 45 to 49 amino acids, and most preferably 47 amino acids. In the case of using ZFN, the first DNA binding domain and the first DNA cleavage domain in the first nuclease subunit are linked by a polypeptide consisting of from 0 to 20 amino acids or from 2 to 10 amino acids, preferably from 3 to 9 amino acids, more preferably from 4 to amino acids and even more preferably from 5 to 7 amino acids. In the case of using FokI-dCas9, the dCas9 and FokI in the first nuclease subunit are linked by a polypeptide consisting of from 1 to 20 amino acids, from 1 to 15 amino acids or from 1 to 10 amino acids, preferably from 2 to 8 amino acids, more preferably from 3 to 7 amino acids, even more preferably from 4 to 6 amino acids, and most preferably amino acids. The same holds for the second nuclease subunit. The first nuclease subunit linked by such a length of polypeptide has high specificity to the length of the moiety including the region formed of the first nucleotide sequence and the region formed of the second nucleotide sequence, and specifically cleaves a spacer region having a specific length. Thus, the nucleic acid is not frequently inserted into a site outside the target site by nonspecific cleavage, and the nucleic acid joined by microhomology-mediated end joining as described later is not frequently cleaved again. This is preferable.

In the nucleic acid contained in the cell into which the vector provided by the second aspect of the present invention is to be inserted, the region formed of the first nucleotide sequence is located between the region formed of the nucleotide sequence recognized by the first DNA binding domain and the region formed of the nucleotide sequence recognized by the second DNA binding domain. Further, the region formed of the second nucleotide sequence is located between the region formed of the nucleotide sequence recognized by the first DNA binding domain and the region formed of the nucleotide sequence recognized by the second DNA binding domain. Further, the predetermined site is located between the region formed of the nucleotide sequence recognized by the first DNA binding domain and the region formed of the nucleotide sequence recognized by the second DNA binding domain. In the nucleic acid, a combination of two nucleotide sequences recognized by the DNA binding domain surrounding the region formed of the first nucleotide sequence may be different from a combination of two nucleotide sequences recognized by the DNA binding domain surrounding the region formed of the second nucleotide sequence. In this case, different nucleases are used as the nuclease for cleaving around the region formed of the first nucleotide sequence and the nuclease for cleaving around the region formed of the second nucleotide sequence. In the nucleic acid contained in the cell, the region formed of the nucleotide sequence recognized by the first DNA binding domain and the region formed of the nucleotide sequence recognized by the second DNA binding domain are separated by a region formed of a nucleotide sequence of preferably from 5 to 40 bases in length, more preferably from 10 to 30 bases in length, and even more preferably from 12 to 20 bases in length. The base length of the region separating both the regions may be the same as or different from the total of the base length of the first nucleotide sequence and the base length of the second nucleotide sequence. For example, in the nucleic acid contained in the cell, in the case where the following conditions are satisfied: the 3′-end of the first nucleotide sequence is directly in contact with the 5′-end of the second nucleotide sequence, there is no overlap between the nucleotide sequence recognized by the first DNA binding domain and the first nucleotide sequence, and there is no overlap between the second nucleotide sequence and the nucleotide sequence recognized by the second DNA binding domain, the base length of the region separating both the regions is the same as the total of the base length of the first nucleotide sequence and the base length of the second nucleotide sequence. However, in the case where one or more items selected from these conditions are not satisfied, the base length of the region separating both the regions is different from the total of the base length of the first nucleotide sequence and the base length of the second nucleotide sequence. The region formed of the first nucleotide sequence in the nucleic acid contained in the cell may partially overlap the region formed of the nucleotide sequence recognized by the first DNA binding domain. Further, the region formed of the second nucleotide sequence in the nucleic acid contained in the cell may partially overlap the region formed of the nucleotide sequence recognized by the second DNA binding domain. In the case where there is a partial overlap, the overlapping moiety consists of a nucleotide sequence of preferably from 1 to 6 bases in length, more preferably from 1 to 5 bases in length, and even more preferably from 2 to 4 bases in length. In the case where there is a partial overlap, the length of a moiety which separates two regions recognized by the DNA binding domain and includes the region formed of the first nucleotide sequence and the region formed of the second nucleotide sequence is greatly reduced by microhomology-mediated end joining as described later. Thus, the linked nucleic acid is hardly cleaved again and the inserted nucleic acid is more stably maintained. This is preferable.

In the vector provided by the second aspect of the present invention, the region formed of the first nucleotide sequence and the region formed of the second nucleotide sequence are each located between the region formed of the nucleotide sequence recognized by the first DNA binding domain and the region formed of the nucleotide sequence recognized by the second DNA binding domain. In the vector, a combination of two nucleotide sequences recognized by the DNA binding domain surrounding the region formed of the first nucleotide sequence may be different from a combination of two nucleotide sequences recognized by the DNA binding domain surrounding the region formed of the second nucleotide sequence. In this case, different nucleases are used as the nuclease for cleaving around the region formed of the first nucleotide sequence and the nuclease for cleaving around the region formed of the second nucleotide sequence. In the vector, the region formed of the nucleotide sequence recognized by the first DNA binding domain may be present at the 5′-end or the 3′-end as compared to the region formed of the nucleotide sequence recognized by the second DNA binding domain. However, in the vector, the nucleotide sequence that is located at the 3′-end of the first nuclease sequence and recognized by the first DNA binding domain or the second DNA binding domain is preferably different from the sequence that is located at the 3′-end of the second nucleotide sequence in the nucleic acid contained in the cell and recognized by the first DNA binding domain or the second DNA binding domain. Further, in the vector, the nucleotide sequence that is located at the 5′-end of the second nuclease sequence and recognized by the first DNA binding domain or the second DNA binding domain is preferably different from the sequence that is located at the 5′-end of the first nucleotide sequence in the nucleic acid contained in the cell and recognized by the first DNA binding domain or the second DNA binding domain. In these cases, the frequency of cleavage occurring again after insertion of a desired nucleic acid can be further reduced by using a nuclease including a heterodimeric DNA cleavage domain in combination. In the vector, one site may be cleaved, or two or more sites may be cleaved by one or more nucleases containing a first DNA binding domain and a second DNA binding domain. The vector cleaved at two sites is, for example, a vector including a region formed of a nucleotide sequence recognized by a first DNA binding domain, a region formed of a first nucleotide sequence, a region formed of a nucleotide sequence recognized by a second DNA binding domain, a desired nucleic acid to be inserted into a cell, the region formed of the nucleotide sequence recognized by the first DNA binding domain, a region formed of a second nucleotide sequence, and the region formed of the nucleotide sequence recognized by the second DNA binding domain in the stated order in the 5′-end to 3′-end direction. In the case of using the vector cleaved at two sites, unnecessary nucleic acids contained in the vector can be removed by nuclease cleavage. Consequently, it is possible to more safely obtain a desired cell containing no unnecessary nucleic acids.

In the vector provided by the second aspect of the present invention, the region that separates the region formed of the nucleotide sequence recognized by the first DNA binding domain from the region formed of the nucleotide sequence recognized by the second DNA binding domain and that includes the region formed of the first nucleotide sequence or the region formed of the second nucleotide sequence consists of a nucleotide sequence of preferably from 5 to 40 bases in length, more preferably from 10 to 30 bases in length, and even more preferably from 12 to 20 bases in length. In the case where the region that separates the region formed of the nucleotide sequence recognized by the first DNA binding domain from the region formed of the nucleotide sequence recognized by the second DNA binding domain includes both the first nucleotide sequence and the second nucleotide sequence, the base length of the region separating both the regions is the same or almost the same as the total of the base length of the first nucleotide sequence and the base length of the second nucleotide sequence. As described above, the first nucleotide sequence is preferably from 3 to 10 bases in length, more preferably from 4 to 8 bases in length, and even more preferably from 5 to 7 bases in length. As described above, the second nucleotide sequence is preferably from 3 to 10 bases in length, more preferably from 4 to 8 bases in length, and even more preferably from 5 to 7 bases in length. In the case where there is an overlap between the region formed of the first nucleotide sequence or the second nucleotide sequence and the region formed of the nucleotide sequence recognized by the DNA binding domain, the case where there is an overlap between the region formed of the first nucleotide sequence and the region formed of the second nucleotide sequence or the case where the region formed of the first nucleotide sequence is not directly linked to the region formed of the second nucleotide sequence, the base length of the region separating both the regions is not the same but almost the same as the total of the base length of the first nucleotide sequence and the base length of the second nucleotide sequence.

In the vector provided by the first or second aspect of the present invention, for example, the first nucleotide sequence in the nucleic acid contained in the cell and the first nucleotide sequence in the vector are joined by microhomology-mediated end joining (MMEJ), and the second nucleotide sequence in the nucleic acid contained in the cell and the second nucleotide sequence in the vector are joined by MMEJ, whereby the desired nucleic acid is inserted into a predetermined site in the nucleic acid contained in the cell.

In the vector provided by the second aspect of the present invention, for example, the nuclease is a homodimeric nuclease and the vector is a circular vector.

In the vector provided by the first aspect of the present invention, for example, the nuclease is an RNA-guided nuclease such as a nuclease based on the CRISPR/Cas system. Preferably, the nuclease is a Cas9 nuclease.

In the vector provided by the second aspect of the present invention, the nuclease is preferably a ZFN, a TALEN or FokI-dCas9, and more preferably a TALEN. The ZFN, TALEN or FokI-dCas9 may be homodimeric or heterodimeric. The nuclease is preferably a homodimeric ZFN, TALEN or FokI-dCas9, and more preferably a homodimeric TALEN.

The nucleases also include their mutants. Such a mutant may be any mutant as long as it exhibits the activity of the nuclease. The mutant is, for example, a nuclease containing the amino acid sequence in which several amino acids, such as 1, 2, 3, 4, 5, 6, 7, 8, 9, 10, 15, 20, 25, 30, 35, 40, 45 or 50 amino acids are substituted, deleted and/or added in the amino acid sequence of the nuclease.

A desired nucleic acid contained in the vector provided by the present invention is, for example, from 10 to 10000 bases in length and may be several kilo bases in length. The desired nucleic acid may also contain a nucleic acid encoding a gene. The gene encoded can be any gene. Examples thereof include genes encoding an enzyme converting a chemiluminescence substrate such as alkaline phosphatase, peroxidase, chloramphenicol acetyltransferase and galactosidase. The desired nucleic acid may contain a nucleic acid encoding a gene capable of detecting the expression level by the light signal. In this case, the presence or absence of the light signal in the cell after vector introduction is detected so that the success or failure of the insertion can be easily confirmed, and the efficiency and frequency of obtaining a cell having a desired nucleic acid inserted therein are improved. Examples of the gene capable of detecting the expression level by the light signal include genes encoding a fluorescent protein such as a green fluorescent protein (GFP), a humanized Renilla green fluorescent protein (hrGFP), an enhanced green fluorescent protein (eGFP), a yellowish green fluorescent protein (mNeonGreen), an enhanced blue fluorescent protein (eBFP), an enhanced cyan fluorescent protein (eCFP), an enhanced yellow fluorescent protein (eYFP) and a red fluorescent protein (RFP or DsRed); and genes encoding a bioluminescence protein such as firefly luciferase and Renilla luciferase.

In the vector provided by the present invention, it is preferable that the region formed of the first nucleotide sequence is directly adjacent to the desired nucleic acid. Further, it is preferable that the desired nucleic acid is directly adjacent to the region formed of the second nucleotide sequence. In the case where the desired nucleic acid contains a functional factor such as a gene, the first and second nucleotide sequences included in the vector may encode a part of the functional factor.

The vector provided by the present invention may be a circular vector or a linear vector. The vector provided by the present invention is preferably a circular vector. Examples of the vector of the present invention include a plasmid vector, a cosmid vector, a viral vector and an artificial chromosome vector. Examples of the artificial chromosome vector include yeast artificial chromosome vector (YAC), bacterial artificial chromosome vector (BAC), P1 artificial chromosome vector (PAC), mouse artificial chromosome vector (MAC) and human artificial chromosome vector (HAC). Examples of the component of the vector include a nucleic acid such as a DNA and an RNA; and a nucleic acid analog such as a GNA, an LNA, a BNA, a PNA and a TNA. The vector may be modified by components other than the nucleic acid, such as saccharides.

According to the seventh aspect, the present invention provides a kit for inserting a desired nucleic acid into a predetermined site in a nucleic acid contained in a cell. The kit according to the seventh aspect of the present invention comprises the vector according to any one of the first to sixth aspects. The kit according to the seventh aspect of the present invention further comprises a vector for expressing a nuclease. The vector for expressing a nuclease is, for example, a vector for expressing a nuclease including a first DNA binding domain and a second DNA binding domain. Examples of the vector for expressing a nuclease include a plasmid vector, a cosmid vector, a viral vector and an artificial chromosome vector. The vector for expressing a nuclease is, for example, a vector set comprising a first vector that contains a gene encoding a first nuclease subunit including a first DNA binding domain and a first DNA cleavage domain and a second vector that contains a gene encoding a second nuclease subunit including a second DNA binding domain and a second DNA cleavage domain. Another example is a vector including both of the gene encoding the first nuclease subunit and the gene encoding the second nuclease subunit. The first and second vectors may be present in different nucleic acid fragments or identical nucleic acid fragments. In the case where different nucleases are used as the nuclease for cleaving around the region formed of the first nucleotide sequence and the nuclease for cleaving around the region formed of the second nucleotide sequence, the kit of the seventh aspect of the present invention comprises a plurality of the vector sets including first and second vectors. In the case of using the nuclease based on the CRISPR/Cas system as a nuclease, the kit of the seventh aspect of the present invention may comprise: a vector for expressing a gRNA and a nuclease for cleaving around the region formed of the first nucleotide sequence in the vector of the first aspect of the present invention; a vector for expressing a gRNA and a nuclease for cleaving around the region formed of the second nucleotide sequence in the vector of the first aspect of the present invention; and a a vector for expressing gRNA and a nuclease for cleaving a predetermined site in a nucleic acid contained in a cell. The vector for expressing a nuclease based on the CRISPR/Cas system may contain a vector for expressing a gRNA and a vector for expressing Cas9 per one cleavage site. The vector for expressing a gRNA and Cas9 may contain both a gene encoding a gRNA and a gene encoding Cas9. Alternatively, the vector may be a vector set including a vector containing the gene encoding a gRNA and a vector containing the gene encoding Cas9. A plurality of vectors having different functions may be present in identical nucleic acid fragments or may be present in different nucleic acid fragments.

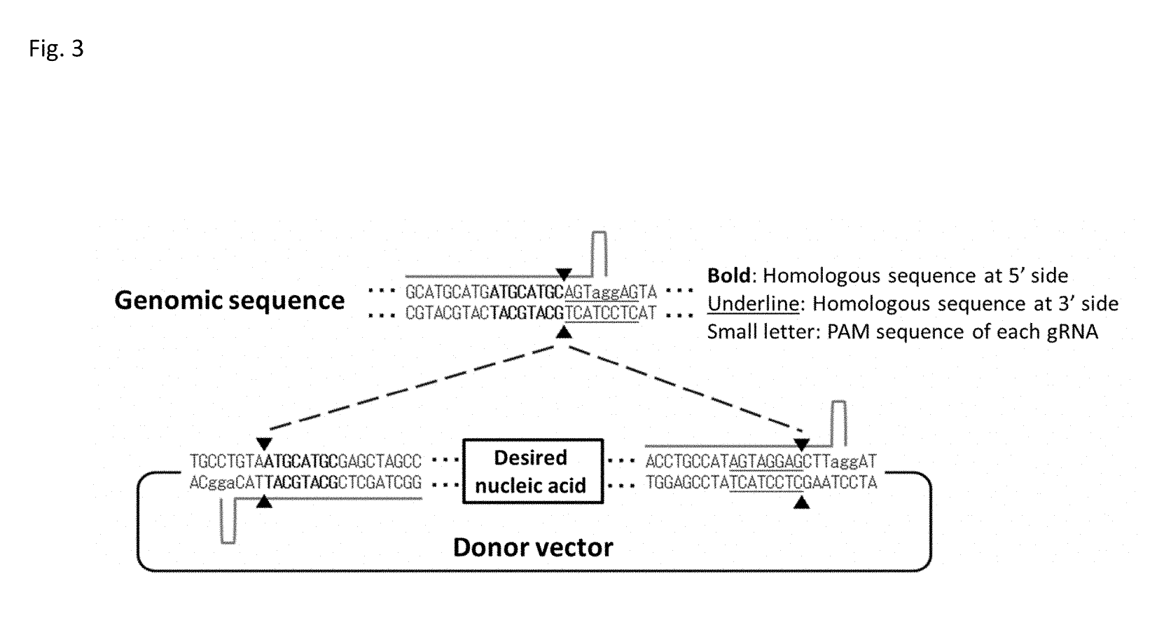

According to the eighth aspect, the present invention provides a method for inserting a desired nucleic acid into a predetermined site in a nucleic acid contained in a cell. The method according to the eighth aspect of the present invention comprises a step of introducing the vector according to any one of the first to sixth aspects of the present invention and the vector for expressing a nuclease into a cell. The vector for expressing a nuclease is, for example, a vector for expressing a nuclease including a first DNA binding domain and a second DNA binding domain as described above. Another example is a vector set including a first vector that contains a gene encoding a first nuclease subunit including a first DNA binding domain and a first DNA cleavage domain and a second vector that contains a gene encoding a second nuclease subunit including a second DNA binding domain and a second DNA cleavage domain. These vectors may be introduced into cells by allowing the vectors to be in contact with ex vivo cultured cells, or by administering the vectors into the living body and allowing the vectors to be indirectly in contact with cells present in the living body. These vectors can be introduced into the cells simultaneously or separately. In the case where these vectors are introduced separately into the cells, for example, a vector for expressing a nuclease may be previously introduced into a cell to produce a stable expression cell line or inducible expression cell line of the nuclease, and then, the vector according to any one of the first to sixth aspects of the present invention may be introduced into the produced stable expression cell line or inducible expression cell line. When the step of introduction into a cell is performed, a nuclease (such as the nuclease including the first DNA binding domain and the second DNA binding domain) functions in the cell, resulting in a nucleic acid fragment including a region formed of a first nucleotide sequence, a desired nucleic acid to be inserted into a cell and a region formed of a second nucleotide sequence in the stated order in the 5′-end to 3′-end direction from the vector. The step results in cleavage of a predetermined site in a nucleic acid in a cell. Thereafter, in the cell, the first nucleotide sequence in the nucleic acid in the cell and the first nucleotide sequence in the vector are joined by microhomology-mediated end joining (MMEJ), and the second nucleotide sequence in the nucleic acid in the cell and the second nucleotide sequence in the vector are joined by MMEJ. As a result, a desired nucleic acid is accurately inserted into a predetermined site of a nucleic acid of a cell. In the case of using the vector of the first aspect of the present invention, the nuclease for combination use specifically cleaves the moiety including the region formed of the first nucleotide sequence and the region formed of the second nucleotide sequence in the nucleic acid in the cell before insertion. However, the linked nucleic acid does not contain the moiety because of insertion of the desired nucleic acid. For example, in the case of using the nuclease based on the CRISPR/Cas system, all the gRNA target sequences lose the PAM sequence and the sequence of 3 bases adjacent to the PAM sequence after linkage. Thus, the nucleic acid is not cleaved again by the nuclease present in the cell and is stably retained. Insertion of the desired nucleic acid occurs at high frequency. In this regard, a combination of the vector of the first aspect of the present invention and the CRISPR/Cas system such that the linked nucleic acid loses the PAM sequence or the base adjacent to the PAM sequence can be appropriately designed with reference to the first and second nucleotide sequences included in both the vector and the nucleic acid in the cell as well as the sequences adjacent to these nucleotide sequences. An example of the design is illustrated in a schematic view in FIG. 3. In the case of using the vector of the second aspect of the present invention, the spacer region separating two DNA binding domains in the linked nucleic acid is shorter than that before linkage. Thus, the nucleic acid is not cleaved again by the nuclease present in the cell and is stably retained. Insertion of a desired nucleic acid occurs at high frequency. In this regard, the cleavage activity of the nuclease including a plurality of DNA binding domains depends on the length of the spacer region sandwiched between the regions recognized by the DNA binding domains. The nuclease specifically cleaves a spacer region having a specific length. In the linked nucleic acid, the spacer region separating two DNA binding domains consists of a nucleotide sequence of preferably from 1 to 20 bases in length, more preferably from 2 to 15 bases in length, and even more preferably from 3 to 10 bases in length.

In the present invention, the vector for inserting a desired nucleic acid into a predetermined site in a nucleic acid contained in a cell and the vector for expressing a nuclease may be identical to or different from each other. In the case of using the nuclease based on the CRISPR/Cas system, the vector for inserting a desired nucleic acid into a predetermined site in a nucleic acid contained in a cell, the vector for expressing a nuclease and the vector for expressing a gRNA may be identical to or different from one another.

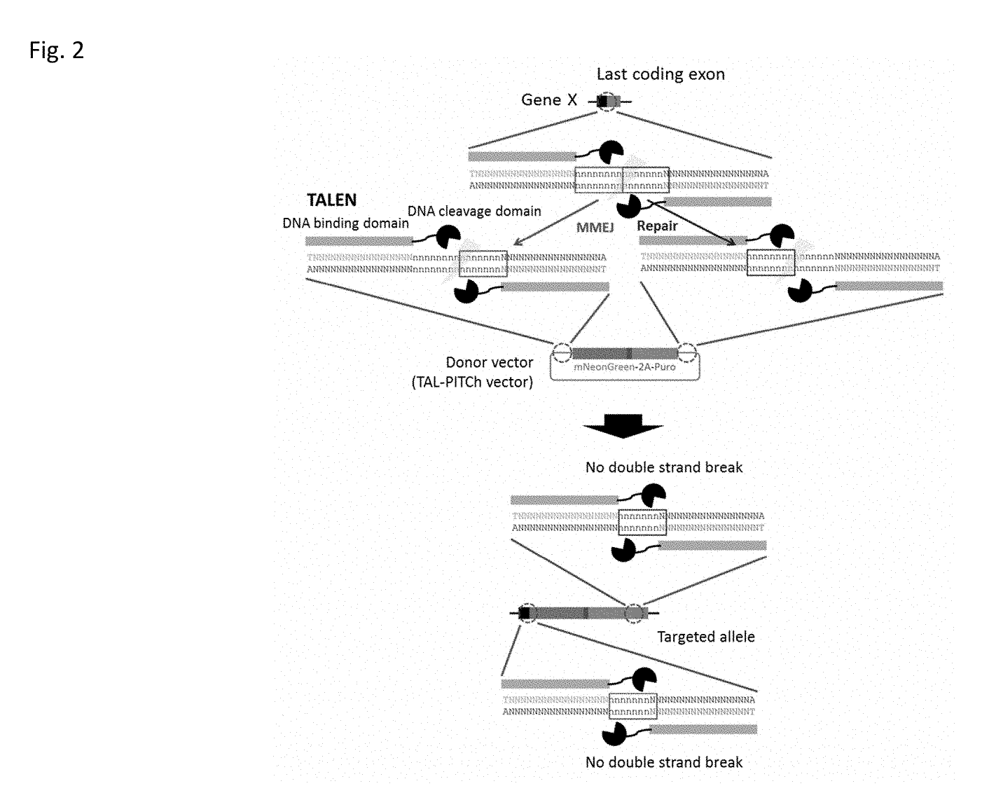

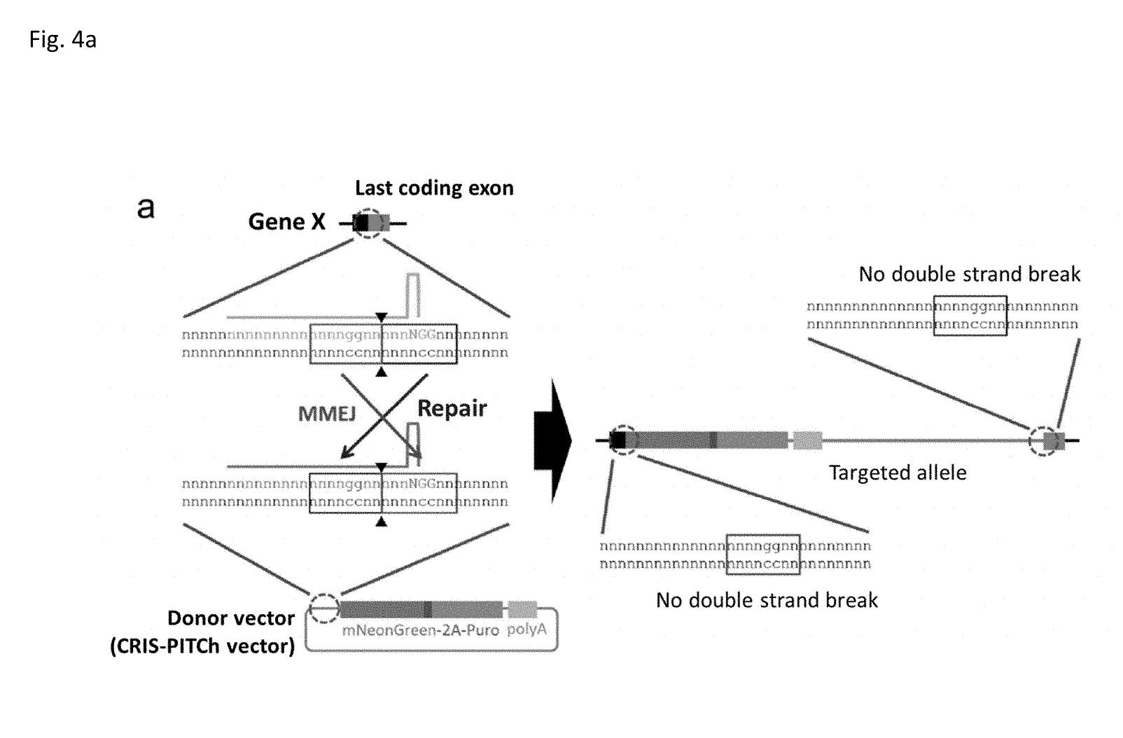

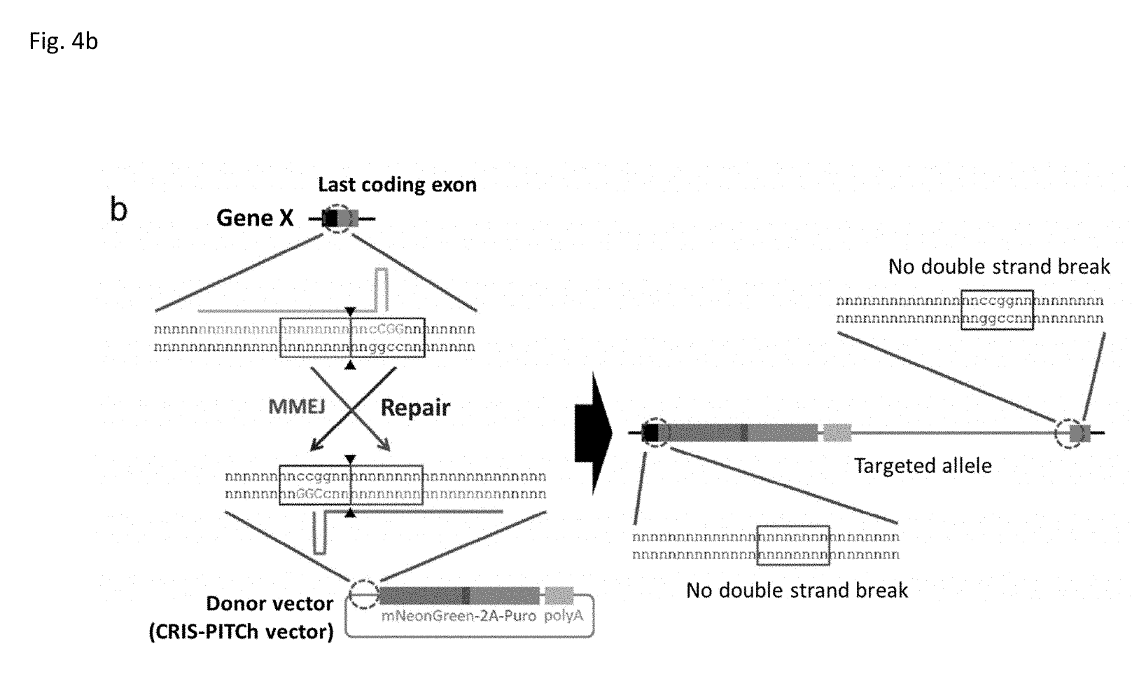

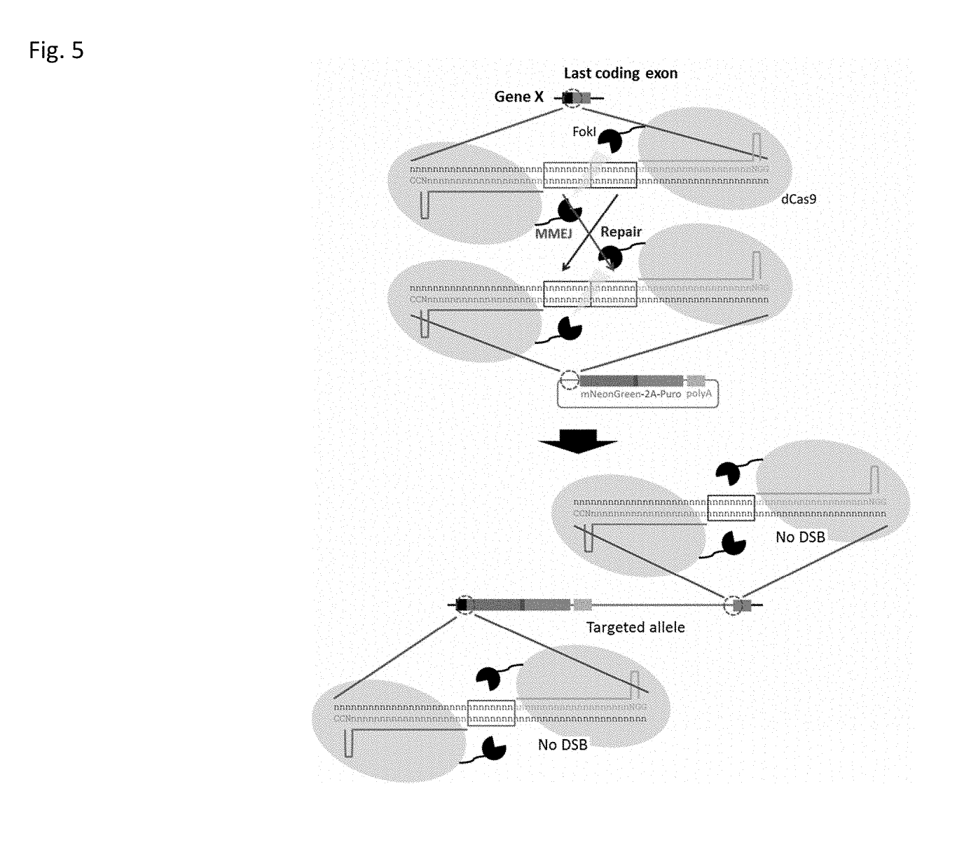

In the case where a desired nucleic acid is inserted using the vector of the present invention, a part of the vector containing a desired nucleic acid may be inserted into a predetermined site in a nucleic acid contained in a cell. Alternatively, the whole vector containing a desired nucleic acid may be inserted into a predetermined site in a nucleic acid contained in a cell. FIG. 1 is a schematic view of a case where the whole vector containing a desired nucleic acid is inserted using TALENs. FIG. 2 is a schematic view illustrating the case where a part of the vector containing a desired nucleic acid is inserted using TALENs. FIG. 3 is a schematic view of a case where a part of the vector containing a desired nucleic acid is inserted into a predetermined site in a nucleic acid contained in a cell using the CRISPR/Cas system. FIGS. 4A and 4B are each a schematic view of a case where the whole vector containing a desired nucleic acid is inserted using the CRISPR/Cas system. FIG. 5 is a schematic view of a case where the whole vector containing a desired nucleic acid is inserted using FokI-dCas9.

According to the ninth aspect, the present invention provides a cell obtained by the method according to the eighth aspect of the present invention. The cell of the ninth aspect of the present invention can be obtained by performing the introduction step in the method of the eighth aspect and then selecting the cell with the nucleic acid inserted. For example, in the case where the nucleic acid to be inserted contains a gene encoding a specific reporter protein, selection of cells can be easily performed at high frequency by detecting the expression of the reporter protein and selecting the amount of the detected expression as an indicator.

According to the tenth aspect, the present invention provides an organism comprising the cell of the ninth aspect of the present invention. In the method of the eighth aspect of the present invention, in the case of administering the vectors into the living body and allowing the vectors to be indirectly in contact with cells present in the living body, the organism of the tenth aspect of the present invention is obtained.

According to the eleventh aspect, the present invention provides a method for producing an organism comprising a desired nucleic acid, comprising a step of differentiating a cell obtained by the method according to the eighth aspect of the present invention. In the method of the eighth aspect of the present invention, a cell comprising a desired nucleic acid is obtained by allowing a vector to be in contact with an ex vivo cultured cell, and differentiating the obtained cell to form an adult organism comprising a desired nucleic acid.

According to the twelfth aspect, the present invention provides an organism produced by the method according to the eleventh aspect of the present invention. The produced organism comprises a desired nucleic acid in a predetermined site of a nucleic acid contained in a cell in the organism, and can be used in various applications such as analysis of the functions of biological substances (e.g., genes, proteins, lipids and saccharides) depending on the function of the desired nucleic acid.

### EXAMPLES

Hereinafter, the present invention will be more specifically described with reference to examples, but the present invention is not limited thereto.

### Example 1

Target Integration with TALEN

In this example, an expression cassette of a fluorescent protein gene was introduced (target integration) into Exon1 of a tyrosinase (tyr) gene of Xenopus laevis using the TALEN and the donor vector (TAL-PITCh vector).

### 1-1. Construction of TALEN:

The TALEN plasmid was constructed in the following manner. A vector constructed by In-Fusion cloning (Clontech Laboratories, Inc.) using pFUS_B6 vector (Addgene) as a template was mixed with a plasmid having a single DNA binding domain. By a Golden Gate reaction, 4 DNA binding domains were linked together (STEP1 plasmid). Thereafter, a vector constructed by In-Fusion cloning (Clontech Laboratories, Inc.) using pcDNA-TAL-NC2 vector (Addgene) as a template was mixed with the STEP1 plasmid. A TALEN plasmid was obtained by the second Golden Gate reaction. The full length sequence of the plasmid is shown in SEQ ID NOs: 1 and 2 (Left_TALEN) and SEQ ID NOs: 3 and 4 (Right_TALEN) of the Sequence Listing.

### 1-2. Construction of Donor Vector for Target Integration (TAL-PITCh Vector):

A plasmid having a modified TALEN sequence in which the first half (first nucleotide sequence) of the spacer of the tyrTALEN target sequence was replaced with the second half (second nucleotide sequence) thereof was constructed (FIG. 1). Inverse PCR was performed with a primer set that adds the above sequence (Xltyr-CMVEGFP-F+Xltyr-CMVEGFP-R; the sequence is shown in Table 1 as described later) using a pCS2/EGFP plasmid with GFP inserted into the ClaI and XbaI sites of pCS2+ as a template. Then, DpnI (New England Biolabs) was added to the PCR reaction solution and the template plasmid was digested. The purified reaction solution was subjected to self ligation, followed by subcloning. A plasmid was prepared from the clone in which accurate insertion was confirmed by sequence analysis and the plasmid was used as a donor vector (The sequence is shown in SEQ ID NOs: 5 and 6 of the Sequence Listing. In SEQ ID NO: 5, the nucleotide sequences 98 to 817 represent an ORF sequence of EGFP. This sequence is inserted into the ClaI/XbaI site of pCS2+. In SEQ ID NO: 5, the nucleotide sequences 1116 to 1167 represent a sequence recognized by the modified TALEN.).

1-3. Microinjection into Xenopus Laevis:

On the day preceding the experiment, human pituitary gonadotrophin (ASKA Pharmaceutical Co., Ltd.) was administered to the male Xenopus laevis and the female Xenopus laevis. The administered units were 150 units (for male) and 600 units (for female). On the next day, several drops of sperm suspension was added to the collected eggs and the eggs were artificially inseminated. After about 20 minutes, a 3% cysteine solution was added to allow the fertilized eggs to be dejellied. Then, the resulting eggs were washed several times with 0.1×MMR (ringer solution for amphibians) and transferred into 5% Ficoll/0.3×MMR. The tyrosinase TALEN mRNA mix (Left, Right 250 pg each) and donor vector (100 pg) constructed in the sections 1-1 and 1-2 were co-introduced into the fertilized eggs by the microinjection method (experimental group). As a negative control, only the TALEN mRNA Right (250 pg) and donor vector (100 pg) were co-introduced. Embryos were cultured at 20° C. and transferred into 0.1×MMR at the blastula stage to facilitate their development.

### 1-4. Detection of Target Integration:

The embryos (at the tadpole stage) into which the TALEN and vector were co-introduced were observed under a fluorescence stereoscopic microscope and the presence or absence of GFP fluorescence was determined. A genomic DNA for each individual was extracted from the embryos of the control and experimental groups. The introduction of the donor vector into the target site was determined by PCR. The junctions between the genome and the 5′- or 3′-side of the vector were amplified by PCR using the primer set designed at the upstream and downstream of the TALEN target sequence and the vector side. The primer set of tyr-genomic-F and pCS2-R was used for the 5′-side, and the primer set of tyr-genomic-R and pCS2-F was used for the 3′-side (the sequence is shown in Table 1 as described later). After agarose electrophoresis confirmation, a band of the target size was cut out and subcloned into pBluescript SK. The inserted sequence was amplified by colony PCR, followed by analysis by direct sequencing. The sequencing was performed using CEQ-8000 (Beckman Coulter, Inc.).

### Result:

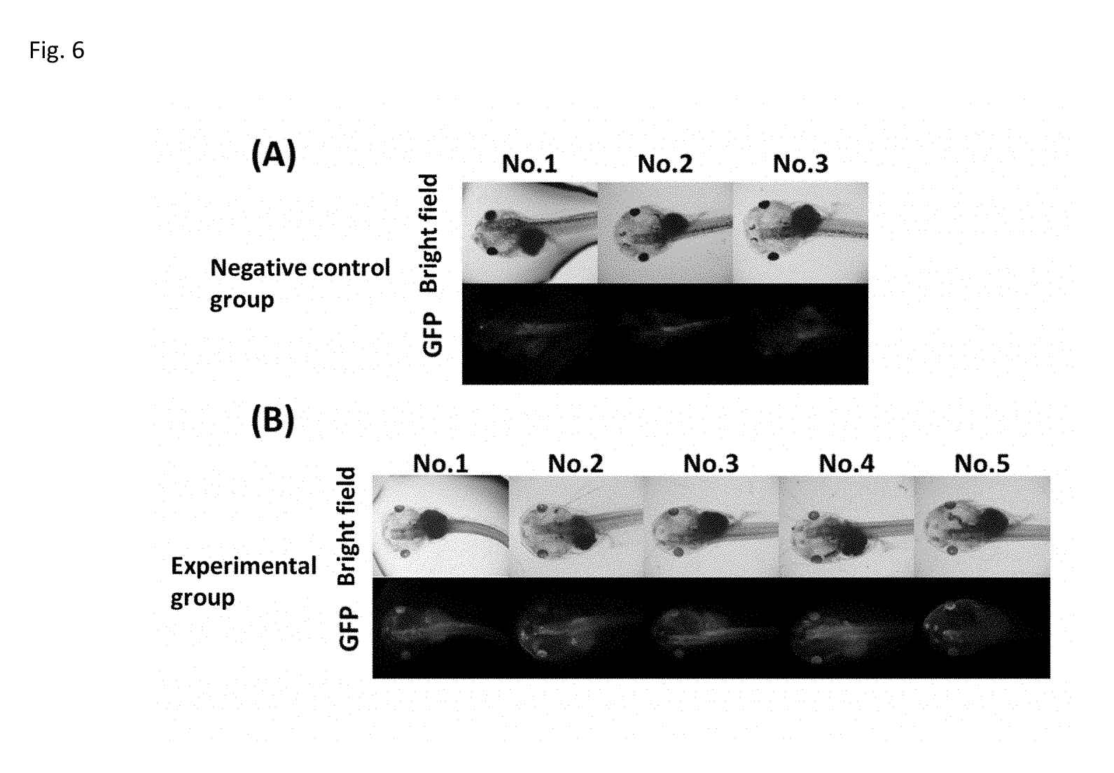

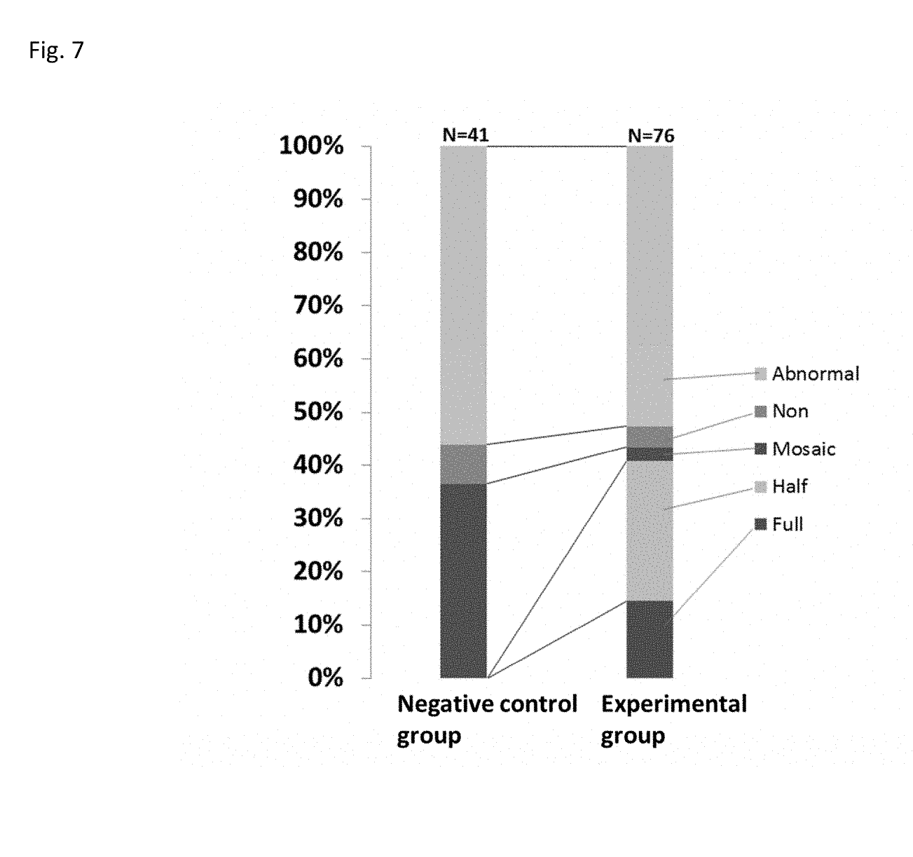

As for the embryos (at the tadpole stage) into which the donor vector was introduced, items A and B in FIG. 6 show phenotypes of the experimental group (TALEN mix+vector-injected embryo) and the negative control group (TALEN R+vector-injected embryo). In the experimental group, the tyr gene was broken and thus an albino phenotype was exhibited in the retinal pigment epithelium and melanophores. Additionally, many individuals generating strong GFP fluorescence throughout the body were observed (item B in FIG. 6). No albino was observed in the negative control group. Individuals generating mosaic GFP fluorescence were partially observed (item A in FIG. 6). The ratio of the phenotypes in the experimental group and the negative control group was classified into four groups: Full: the individuals in which GFP fluorescence is observed in the whole body; Half: the individuals in which half of the right or left side has fluorescence; Mosaic: the individuals with mosaic fluorescence; and Non: the individuals in which GFP fluorescence is not observed (FIG. 7). The individuals of Full and Half were not observed in the negative control group, meanwhile, about 20% of the survived individuals exhibited phenotypes of Full and about 50% of the survived individuals exhibited phenotypes of Half in the experimental group.

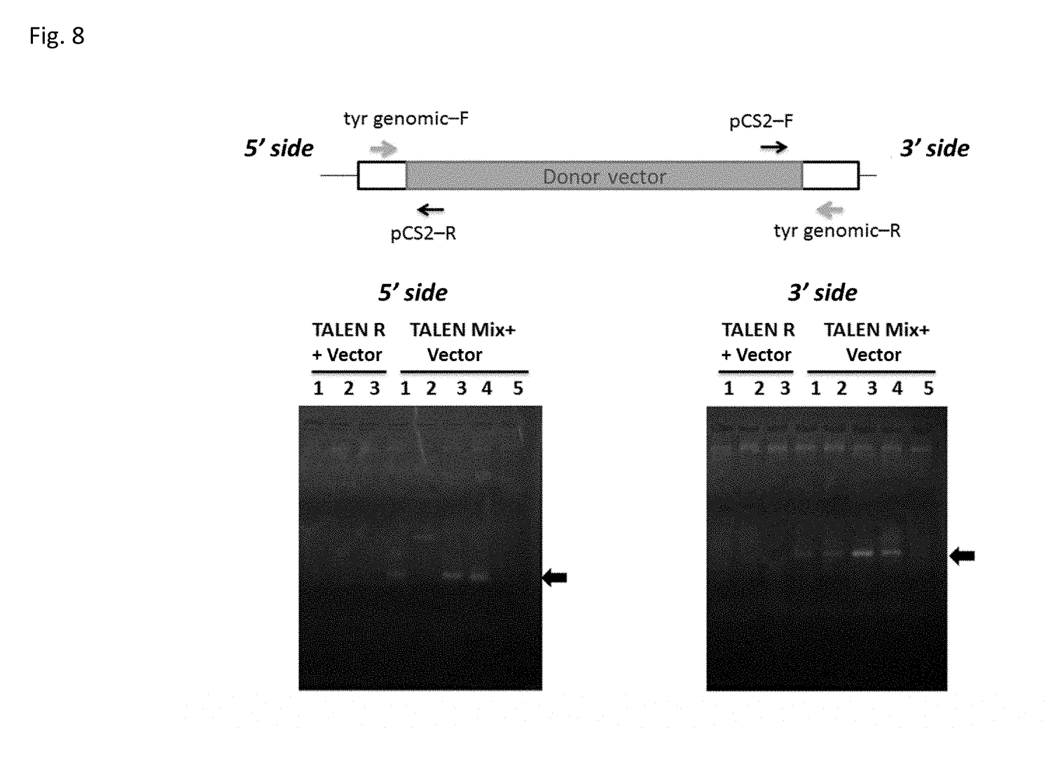

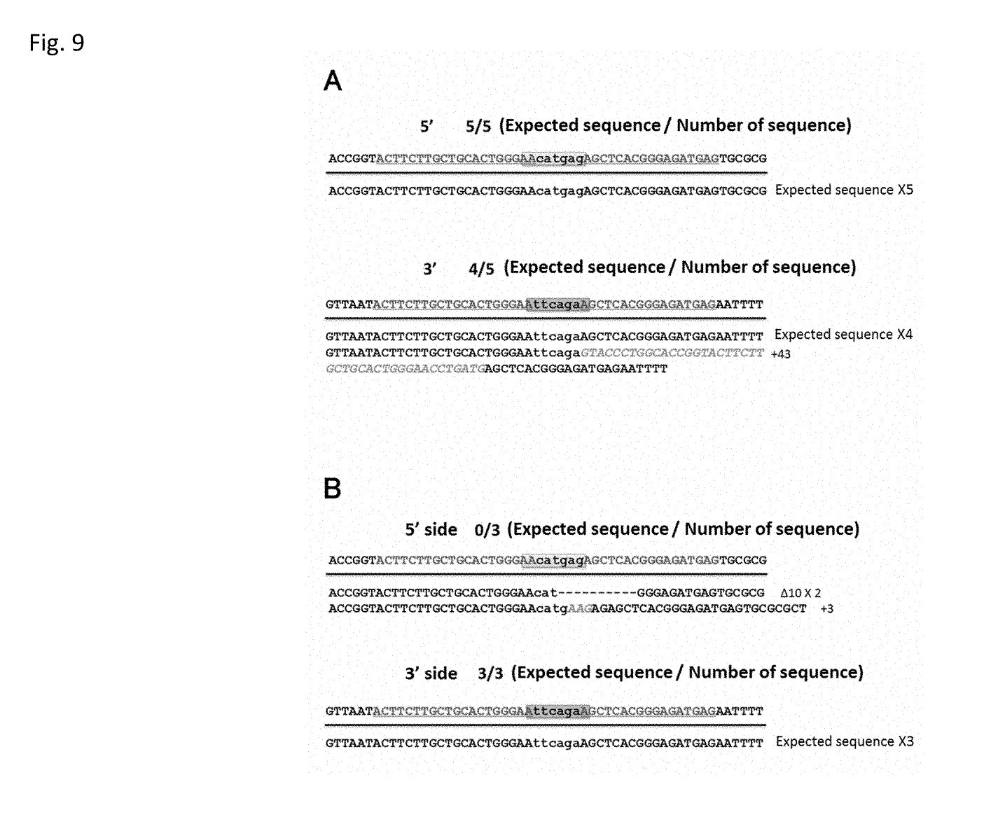

Subsequently, a genomic DNA was respectively extracted from 5 tadpoles exhibiting phenotypes of Full and 3 individuals of the negative control group observed in FIG. 6, followed by genotyping. In order to confirm the inserted portion on the genome and the junction of the vector, the junctions between the target site and the 5′- or 3′-side of the donor vector were amplified by PCR using the primer set designed at the upstream and downstream of the tyrTALEN target sequence and the vector side (FIG. 8). The PCR products were subjected to electrophoresis and bands having an estimated size were confirmed in the experimental group Nos. 1, 3 and 4 (at the 5′-side) and the experimental group Nos. 2, 3 and 4 (at the 3′-side) (FIG. 8, indicated by arrows). On the other hand, no PCR product was confirmed in the negative control group. Then, in order to examine the sequence of the junctions, the PCR products at the 5′- and 3′-sides detected in Nos. 3 and 4 were subcloned, followed by sequence analysis. As a result, the sequence expected in the case of being joined by MMEJ was confirmed at a ratio of 100% (5/5 clone) in the junction at the 5′-side in No. 3, meanwhile, the sequence expected was confirmed at a ratio of 80% (4/5 clone) in the junction at the 3′-side (FIG. 9A). The sequence with 10 bases deleted or 3 bases inserted was confirmed in the junction at the 5′-side in No. 4, meanwhile, the sequence expected was confirmed at a ratio of 100% (3/3) in the junction at the 3′-side (FIG. 9B).

The sequences of the primers used in the sections 1-1 to 1-4 are shown in Table 1 below.

TABLE 1

| SEQ | | |
|---|---|---|
| ID | | |
| NO: | Primer name | Sequence (from 5′ to 3′) |
| 7 | Xltyr- | AACATGAGAGCTCACGGGAGATGAGTGCGCG |
| | CMVEGFP-F | CTTGGCGTAATCAT |
| 8 | Xltyr- | TTCTGAATTCCCAGTGCAGCAAGAAGTATTA |
| | CMVEGFP-R | ACCCTCACTAAAGGGA |
| 9 | tyr- | GGAGAGGATGGCCTCTGGAGAGATA |
| | genomic-F | |
| 10 | tyr- | GGTGGGATGGATTCCTCCCAGAAG |
| | genomic-R | |
| 11 | pCS2-F | ATAAGATACATTGATGAGTTTGGAC |
| 12 | pCS2-R | ATGCAGCTGGCACGACAGGTTTCCC |

### Example 2

Target Integration into HEK293T Cell Using CRISPR/Cas9 System

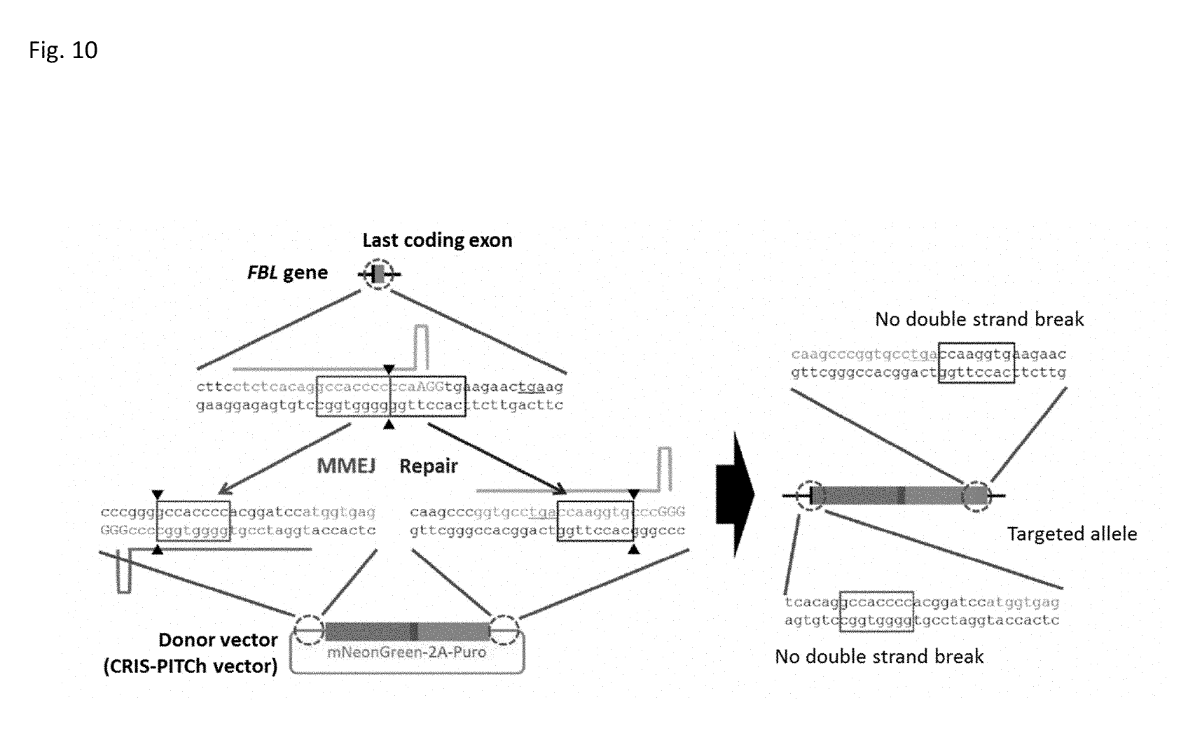

In this example, a fluorescent protein gene expression cassette was introduced (target integration) into the last coding exon of fibrillarin (FBL) gene in a HEK293T cell using the CRISPR/Cas9 system. The outline of this example is illustrated in FIG. 10. Briefly, the vector expressing three types of gRNAs indicated in orange, red and green in FIG. 10 and Cas9 and the donor vector (CRIS-PITCh vector) were co-introduced into the HEK293T cell and the resulting cell was selected by puromycin. Thereafter, DNA sequencing and fluorescent observation were carried out.

2-1. Construction of Vector Expressing gRNA and Cas9:

A vector simultaneously expressing three types of gRNAs, and Cas9 was constructed as described in SCIENTIFIC REPORTS 2014 Jun. 23; 4: 5400. doi: 10.1038/srep05400. Briefly, the pX330 vector (Addgene; Plasmid 42230) was modified so that a plurality of gRNA expression cassettes could be linked by a Golden Gate reaction. The annealed synthetic oligonucleotides were inserted into three types of modified pX330 vectors. Specifically, oligonucleotides 13 and 14 were annealed to each other to produce a synthetic oligonucleotide for forming a genome cleavage gRNA (indicated in orange in FIG. 10). Further, oligonucleotides 15 and 16 were annealed to each other to produce a synthetic oligonucleotide for forming a genome cleavage gRNA at the 5′-side of the donor vector (indicated in red in FIG. 10). Further, oligonucleotides 17 and 18 were annealed to each other to produce a synthetic oligonucleotide for forming a genome cleavage gRNA at the 3′-side of the donor vector (indicated in green in FIG. 10). Each of the produced synthetic oligonucleotides was inserted into each of the plasmids and then the vectors were integrated by a Golden Gate reaction, and a vector simultaneously expressing three types of gRNAs, and Cas9 was obtained.

### 2-2. Construction of Donor Vector for Target Integration (CRIS-PITCh Vector):

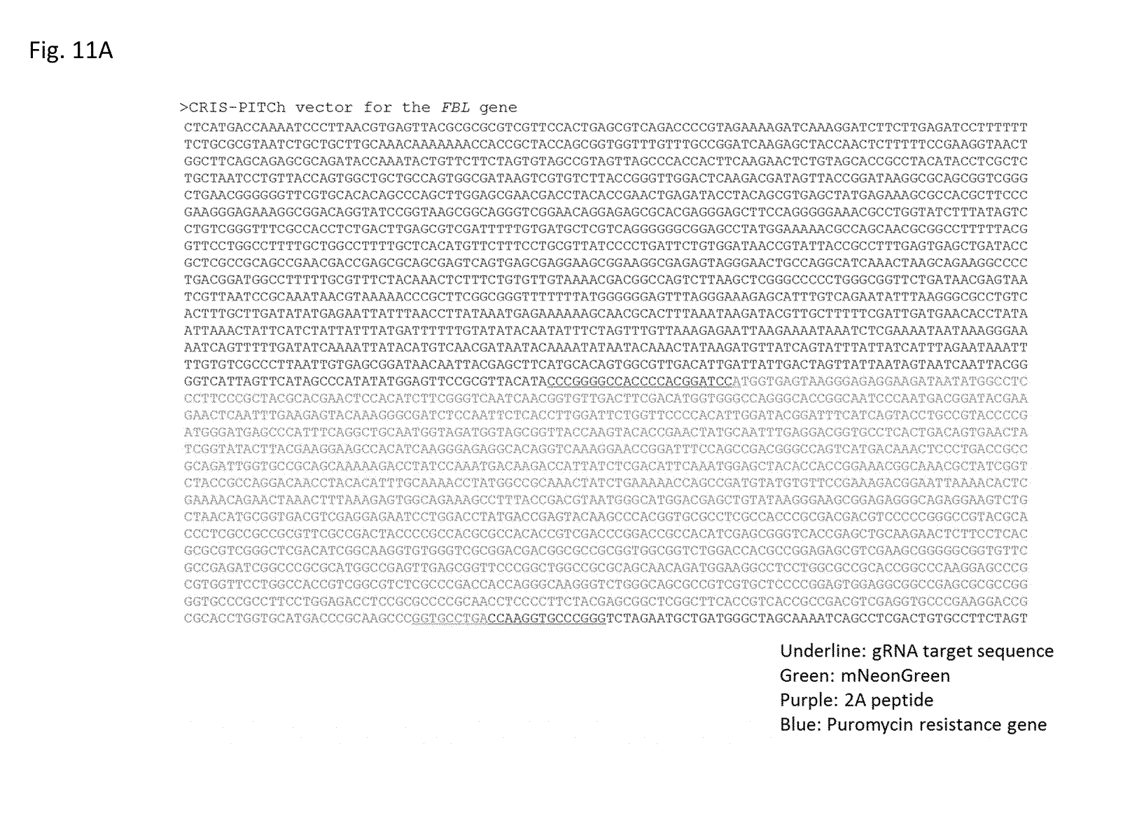

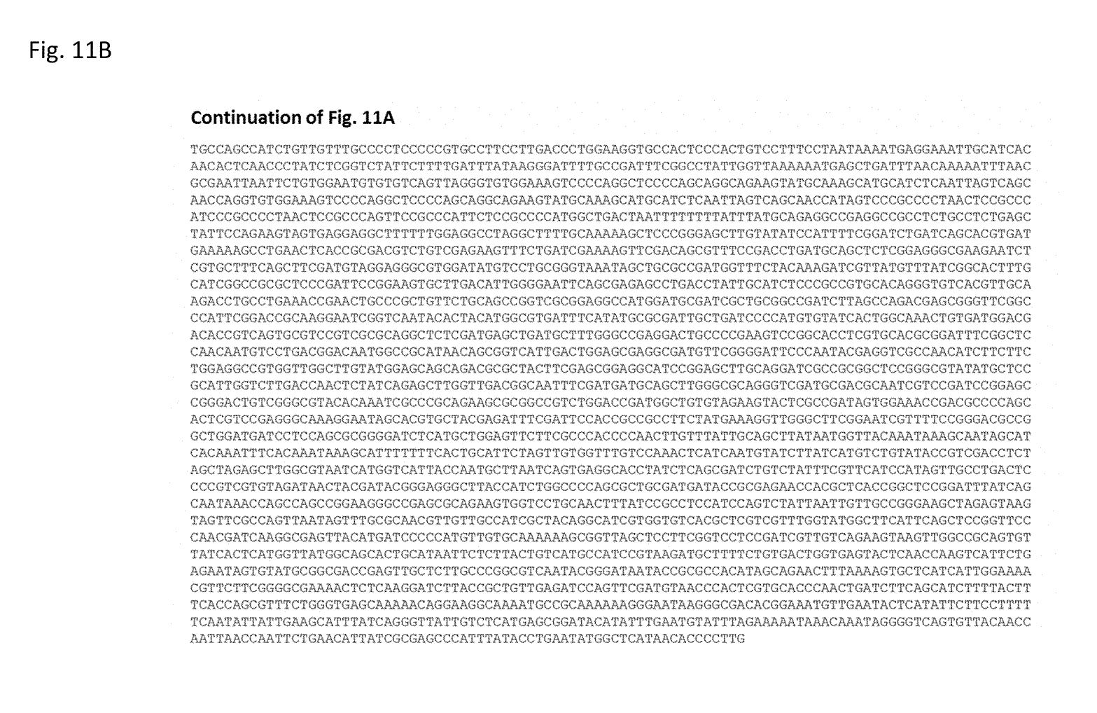

The CRIS-PITCh vector was constructed in the following manner. While a CMV promoter on the vector based on pCMV (Stratagene) was removed, In-Fusion cloning was used to construct a vector such that the gRNA target sequence at the 5′-side, the mNeonGreen coding sequence, the 2A peptide coding sequence, the puromycin resistance gene coding sequence and the gRNA target sequence at the 3′-side were aligned in this order. FIGS. 11A and 11B show the full length sequence (SEQ ID NO: 23) of the constructed vector. In FIGS. 11A and 11B, the mNeonGreen coding sequence is indicated in green (nucleotides 1566 to 2273 of SEQ ID NO: 23), the 2A peptide coding sequence is indicated in purple (nucleotides 2274 to 2336 of SEQ ID NO: 23), and the puromycin resistance gene coding sequence is indicated in blue (nucleotides 2337 to 2936 of SEQ ID NO: 23). The gRNA target sequences at the 5′- and 3′-sides are underlined.

2-3. Introduction into HEK293T Cells:

Introduction into HEK293T cells was performed in the following manner. HEK293T cells were cultured in 10% fetal bovine serum-containing Dulbecco's modified Eagle's medium (DMEM). The cultured cells were seeded at a density of 1×10⁵ cells per well on a 6-well plate on the day before the introduction of plasmids. In the introduction of plasmids, 400 ng of a vector expressing a gRNA and Cas9 and 200 ng of a CRIS-PITCh vector were introduced using Lipofectamine LTX (Life Technologies). After the introduction of plasmids, the cells were cultured in a drug-free medium for 3 days and then cultured in a culture medium containing 1 μg/mL of puromycin for 6 days. Thereafter, the cultured cells were single-cell cloned on a 96-well plate by limiting dilution.

### 2-4. Detection of Target Integration:

The HEK293T cell into which the vector expressing a gRNA and Cas9 and the CRIS-PITCh vector were co-introduced was observed using a confocal laser scanning microscope, and the presence or absence of fluorescence was determined. Then, the genomic DNA was extracted from a clone of puromycin resistant cells and the introduction of the donor vector into the target site was confirmed. The junctions between the genome and the 5′- or 3′-side of the vector were amplified by PCR using the primer set designed at the upstream and downstream of the CRISPR target sequence. The primer set of primers 19 and 20 was used for the 5′-side, and the primer set of primers 21 and 22 was used for the 3′-side (the sequence is shown in Table 2 as described later). After agarose electrophoresis confirmation, a band of the target size was cut out and analyzed by direct sequencing. The sequencing was performed using ABI 3130xl Genetic analyzer (Life Technologies).

### Result:

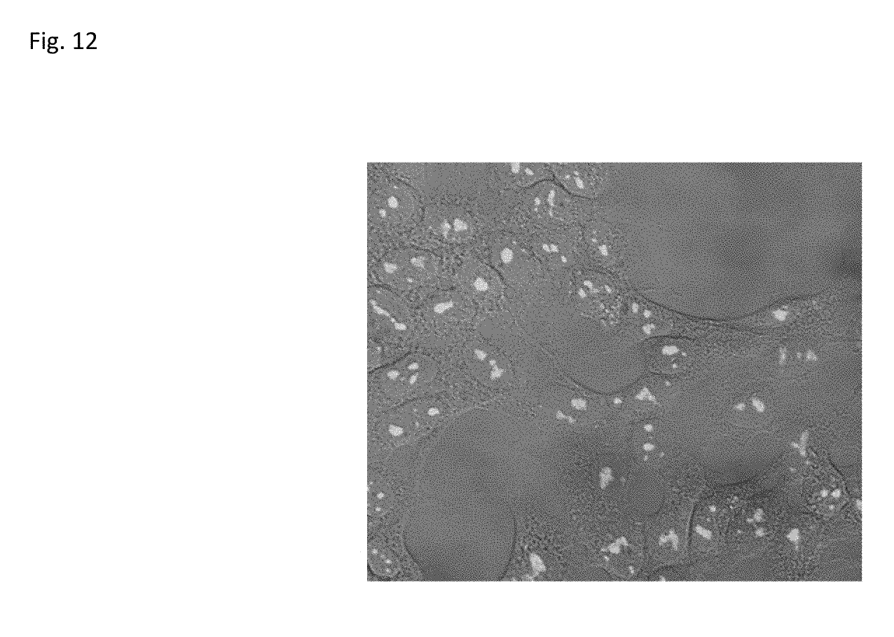

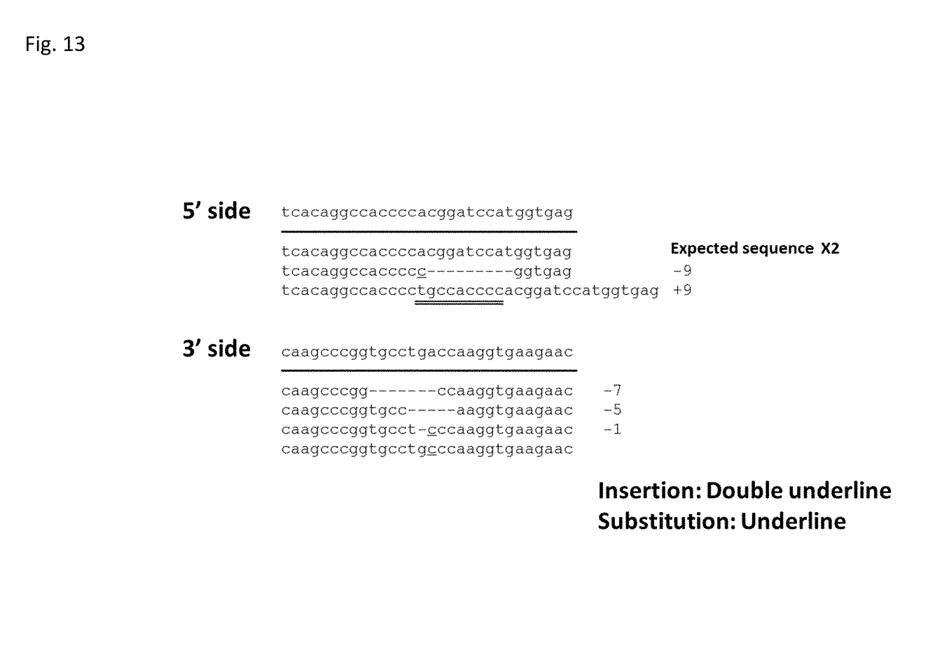

The result observed with the confocal laser scanning microscope is shown in FIG. 12. FBL is a protein specific to nucleoli. Accordingly, in the case where the target integration of the fluorescent protein gene to the FBL gene is successful, the fluorescent protein is localized in the nucleoli. As shown in FIG. 12, a fluorescence image corresponding to the localization pattern (nucleoli) of the FBL protein was obtained. Subsequently, the sequences of the junctions between the genome and the 5′- or 3′-side of the introduced vector were examined. As a result, the sequence expected when the junction at the 5′-side was joined by MMEJ was present at a ratio of 50% (2/4 clone). The remaining two clones had 9 bases deleted or inserted (FIG. 13). The completely expected sequence in the junction at the 3′-side was present at 0% (0/4 clone), but the sequence in which only one base was substituted was present (1 clone). In addition, it was confirmed that one clone had one base deleted, one clone had 5 bases deleted, and one clone had 7 bases deleted (FIG. 13). Similarly, when the fluorescent protein gene expression cassette was introduced into a β-actin (ACTB) locus of HCT116 cells using the CRISPR/Cas9 system (target integration), the same result as that of the target integration into the HEK293T cell was obtained.

The sequences of the oligonucleotides used in the sections 2-1 to 2-4 are shown in Table 2 below.

TABLE 2
SEQ
ID

| NO: | Name | Sequence (from 5′ to 3′) |
|---|---|---|
| 13 | Oligonucleotide 13 | CACCGCTCTCACAGGCCACCCCCCA |
| 14 | Oligonucleotide 14 | AAACTGGGGGGTGGCCTGTGAGAGC |
| 15 | Oligonucleotide 15 | CACCGTGGATCCGTGGGGTGGCCCC |
| 16 | Oligonucleotide 16 | AAACGGGGCCACCCCACGGATCCAC |
| 17 | Oligonucleotide 17 | CACCGGTGCCTGACCAAGGTGCCC |
| 18 | Oligonucleotide 18 | AAACGGGCACCTTGGTCAGGCACC |
| 19 | Primer 19 | ACACCAAGACAGACATCTCTGTCCC |
| | | TTG |
| 20 | Primer 20 | ATCCGTATCCAATGTGGGGAAC |
| 21 | Primer 21 | CCGCAACCTCCCCTTCTACGAG |
| 22 | Primer 22 | TCAGCAGGTCAAGGGGAGGAATG |

1. A vector for inserting a desired nucleic acid into a predetermined site in a nucleic acid contained in a cell by a nuclease,
wherein the nucleic acid contained in the cell includes a region formed of a first nucleotide sequence, the predetermined site, and a region formed of a second nucleotide sequence in the stated order in a 5′-end to 3′-end direction,
wherein the nuclease specifically cleaves a moiety including the region formed of the first nucleotide sequence and the region formed of the second nucleotide sequence included in the cell, and
wherein the vector includes a region formed of a first nucleotide sequence, the desired nucleic acid, and a region formed of a second nucleotide sequence in the stated order in a 5′-end to 3′-end direction.

2. A vector for inserting a desired nucleic acid into a predetermined site in a nucleic acid contained in a cell by a nuclease including a first DNA binding domain and a second DNA binding domain,
wherein the nucleic acid contained in the cell includes a region formed of a first nucleotide sequence, the predetermined site, and a region formed of a second nucleotide sequence in the stated order in a 5′-end to 3′-end direction,
wherein the region formed of the first nucleotide sequence, the predetermined site and the region formed of the second nucleotide sequence in the nucleic acid contained in the cell are each located between a region formed of a nucleotide sequence recognized by the first DNA binding domain and a region formed of a nucleotide sequence recognized by the second DNA binding domain,
wherein the vector includes a region formed of a first nucleotide sequence, the desired nucleic acid, and a region formed of a second nucleotide sequence in the stated order in a 5′-end to 3′-end direction,
wherein the region formed of the first nucleotide sequence and the region formed of the second nucleotide sequence in the vector are each located between a region formed of a nucleotide sequence recognized by the first DNA binding domain and a region formed of a nucleotide sequence recognized by the second DNA binding domain, and
wherein the vector produces a nucleic acid fragment including the region formed of the first nucleotide sequence, the desired nucleic acid, and the region formed of the second nucleotide sequence in the stated order in the 5′-end to 3′-end direction by the nuclease.

3. The vector according to claim 1 or 2, wherein the first nucleotide sequence in the nucleic acid contained in the cell and the first nucleotide sequence in the vector are joined by microhomology-mediated end joining, and the second nucleotide sequence in the nucleic acid contained in the cell and the second nucleotide sequence in the vector are joined by microhomology-mediated end joining, whereby the desired nucleic acid is inserted.

4. The vector according to claim 2, wherein the nuclease is a homodimeric nuclease and the vector is a circular vector.

5. The vector according to claim 1, wherein the nuclease is a Cas9 nuclease.

6. The vector according to claim 2, wherein the nuclease is a TALEN.

7. A kit for inserting a desired nucleic acid into a predetermined site in a nucleic acid contained in a cell, comprising the vector according to any one of claims 1 to 6 and a vector for expressing a nuclease.

8. A method for inserting a desired nucleic acid into a predetermined site in a nucleic acid contained in a cell, comprising a step of introducing the vector according to any one of claims 1 to 6 and a vector for expressing a nuclease into a cell.

9. A cell obtained by the method according to claim 8.

10. An organism comprising the cell according to claim 9.

11. A method for producing an organism comprising a desired nucleic acid, comprising a step of differentiating a cell obtained by the method according to claim 8.

12. An organism produced by the method according to claim 11.
